(19) United States
(12) Patent Application Publication
(10) Pub. No.: US 2025/0129428 A1
(43) Pub. Date: Apr. 24, 2025

(54) METHODS AND MATERIALS FOR PREDICTING THE PROGRESSION OF PROSTATE CANCER AND TREATING SAME

(71) Applicant: GOPATH LABORATORIES, LLC, Buffalo Grove, IL (US)

(72) Inventor: Jianfeng XU, Buffalo Grove, IL (US)

(21) Appl. No.: 18/685,129

(22) PCT Filed: Aug. 16, 2022
(86) PCT No.: PCT/US2022/040475

§ 371 Date: Feb. 20, 2024

Related U.S. Application Data
(60) Provisional application No. 63/234,002, filed on Aug. 17, 2021.

(51) Int. Cl. C12Q 1/6886

(52) U.S. Cl. CPC C12Q 1/6886; C12Q 2600/106; C12Q 2600/118; C12Q 2600/156; C12Q 2600/16

(57) ABSTRACT

Methods and materials for predicting prostate cancer progression are provided. Such methods may include obtaining from the subject a biological sample from a prostate cancer; isolating genomic DNA from the biological sample; determining copy number alterations (CNAs) in one or more genes selected from the group consisting of: CHD1, PTEN, CDKN1B, BRCA2, RB1, USP10, SERPINF1, TP53, SERPINB5 and MYC in the genomic DNA; and treating the subject with an anticancer agent based on the CNAs of the one or more genes.

### CROSS-REFERENCE TO RELATED APPLICATION

This application claims the benefit of and priority to U.S. Provisional Patent Application Ser. No. 63/234,002, entitled “METHODS AND MATERIALS FOR PREDICTING THE PROGRESSION OF PROSTATE CANCER AND TREATING SAME,” filed on Aug. 17, 2021, the entire contents of which is incorporated herein by reference.

### FIELD

The present disclosure generally relates to methods and materials for the detection and treatment of prostate cancer.

### BACKGROUND

Prostate cancer (PCa) is the most common noncutaneous cancer among men, with an estimated 191 930 new cases in 2020 (Siegel R L, Miller K D, Jemal A. Cancer statistics, 2020. CA Cancer J Clin. 2020; 70 (1): 7-30). Many prostate tumors are considered to be indolent (nonaggressive) tumors that do not require treatment. However, the subsets of PCa that are aggressive are estimated to account for more than 33,000 deaths in 2020. PCa-specific death is primarily caused by metastasis of the cancer cells harboring genetic alterations of driver genes that can be traced back to the tumors in the prostate (Liu W, Laitinen S, Khan S, et al.

Copy number analysis indicates monoclonal origin of lethal metastatic prostate cancer. Nature Med. 2009; 15 (5): 559-565; Haffner M C, Mosbruger T, Esopi D M, et al. Tracking the clonal origin of lethal prostate cancer. J Clin Invest. 2013; 123 (11): 4918-4922; Hong M K, Macintyre G, Wedge D C, et al. Tracking the origins and drivers of subclonal metastatic expansion in prostate cancer. Nat Commun. 2015; 6:6605). The inability to reliably distinguish between these two forms of the disease, especially at early stages, has resulted in the overtreatment of many patients and undertreatment of others (Delpierre C, Lamy S, Kelly-Irving M, et al. Life expectancy estimates as a key factor in over-treatment: the case of prostate cancer. Cancer Epidemiol. 2013; 37 (4): 462-468; Lee Y J, Park J E, Jeon B R, Lee S M, Kim S Y, Lee Y K. Is prostate-specific antigen effective for population screening of prostate cancer? A systematic review. Ann Lab Med. 2013; 33 (4): 233-241; Moyer V A, U.S. Preventive Services Task Force. Screening for prostate cancer: U.S. Preventive Services Task Force recommendation statement. Ann Intern Med. 2012; 157 (2): 120-134).

Therefore, there exists a need to identify markers that can distinguish between aggressive and non-aggressive types of PCa tumors at the time of diagnosis as well as to more efficiently identify the genes that drive cancer progression.

### SUMMARY

The present disclosure provides methods for treating prostate cancer in a subject in need thereof. Such methods may comprise obtaining from the subject a biological sample from a prostate cancer; isolating genomic DNA from the biological sample; determining copy number alterations (CNAs) in one or more genes selected from the group consisting of: CHD1, PTEN, CDKN1B, BRCA2, RB1, USP10, SERPINF1, TP53, SERPINB5 and MYC in the genomic DNA; and treating the subject with an anticancer agent based on the CNAs of the one or more genes. The probe panel disclosed herein can detect CNAs in tumor DNA at the time of diagnosis or surgery. Furthermore, the CNAs disclosed herein independently predict metastatic/lethal cancer, particularly among men with clinically low-risk disease at diagnosis.

In some embodiments, the CNAs are detected in less than 50 ng of genomic DNA. In a further embodiment, the genomic DNA is from a biological sample comprising greater than or equal to 50% of cancer cells.

In some embodiments, the CNAs are detected in less than 10 ng of genomic DNA. In a further embodiment, the genomic DNA is from a biological sample comprising greater than or equal to 50% of cancer cells.

In some embodiments, the CNAs are detected in 2 ng or less of genomic DNA. In a further embodiment, the genomic DNA is from a biological sample comprising greater than or equal to 50% of cancer cells.

In some embodiments, the biological sample is a formalin-fixed paraffin-embedded tissue.

In some embodiments, the genes are two or more of CHD1, PTEN, CDKN1B, BRCA2, RB1, USP10, SERPINF1, TP53, SERPINB5 and MYC.

In some embodiments, the genes are three or more of CHD1, PTEN, CDKN1B, BRCA2, RB1, USP10, SERPINF1, TP53, SERPINB5 and MYC.

In some embodiments, the genes are four or more of CHD1, PTEN, CDKN1B, BRCA2, RB1, USP10, SERPINF1, TP53, SERPINB5 and MYC.

In some embodiments, the genes consist of only PTEN, MYC, CDKN1B and BRAC2.

In some embodiments, the methods further comprise classifying the subject as a progressor or a non-progressor based on the determination in c).

The present disclosure also provides a panel of probes for the prediction of aggressive prostate cancer, the panel comprising: probes that specifically hybridize to copy number alterations (CNAs) in one or more genes selected from the group consisting of: CHD1, PTEN, CDKN1B, BRCA2, RB1, USP10, SERPINF1, TP53, SERPINB5 and MYC.

In some embodiments, the genes are two or more of CHD1, PTEN, CDKN1B, BRCA2, RB1, USP10, SERPINF1, TP53, SERPINB5 and MYC.

In some embodiments, the genes are three or more of CHD1, PTEN, CDKN1B, BRCA2, RB1, USP10, SERPINF1, TP53, SERPINB5 and MYC.

In some embodiments, the genes are four or more of CHD1, PTEN, CDKN1B, BRCA2, RB1, USP10, SERPINF1, TP53, SERPINB5 and MYC.

In some embodiments, the genes consist of only PTEN, MYC, CDKN1B and BRAC2.

In some embodiments, the methods disclosed herein may further comprise detecting the expression of one or more of the genes selected from the group consisting of: ABCC4 (NM_001105515.2), ACACA (NM_198834.1), ACSM1 (NM_052956.2), AMACR (NM_014324.4), APOC1 (NM_001645.5), APOE (NM_000041.2), ARHGEF26 (NM_001251963.1), BGN (NM_001711.3), BIRC5 (NM_001168.2), CDC20 (NM_001255.2), CDK1 (NM_001786.4), CENPF (NM_016343.3), COL10A1 (NM_000493.3), COL1A1 (NM_000088.3), DAPK1 (NM_001288729.1), DBNDD1 (NM_001042610.1), DLGAP5 (NM_014750.4), EPCAM (NM_002354.1), EZH2 (NM_001203247.1), FOXM1 (NM_202002.1), GJB1 (NM_000166.5), GLYATL1 (NM_001220494.1), GOLM1 (NM_016548.3), HMMR (NM_012484.2), HOXC4 (NM_014620.4), HOXC6 (NM_153693.3), HPN (NM_182983.1), MTHFD2 (NM_006636.3), MYBPC1 (NM_002465.2), MYO6 (NM_004999.3), NUSAP1 (NM_016359.4), PCA3 (NR_015342.1), PCAT7 (NR_121566.2), PCCB (NM_000532.4), PCLAF (NM_014736.5), PLA2G7 (NM_001168357.1), PRC1 (NM_199413.1), PYCR1 (NM_006907.2), RAB17 (NM_022449.3), RAP1GAP (NM_002885.1), RRM2 (NM_001034.1), SAPCD2 (NM_178448.3), SMPDL3B (NM_001009568.1), THBS2 (NM_003247.3), TK1 (NM_003258.1), TMEM238 (NM_001190764.1), TOP2A (NM_001067.3), TPX2 (NM_012112.4), UBE2C (NM_181803.1), ZWILCH (NM_017975.4), ABCF1, (NM_001090.2), G6PD (NM_000402.4), and TBP (NM_001172085.1).

The present disclosure also provides methods for predicting prostate cancer progression in a subject in need thereof, the method comprising: obtaining from the subject a biological sample from a prostate cancer; isolating genomic DNA from the biological sample; determining copy number alterations (CNAs) in one or more genes selected from the group consisting of: CHD1, PTEN, CDKN1B, BRCA2, RB1, USP10, SERPINF1, TP53, SERPINB5 and MYC in the genomic DNA; and classifying the subject as a progressor or a non-progressor based on the determination.

In some embodiments, the prostate cancer is or will become metastatic and/or lethal if the subject is classified as a progressor.

In some embodiments, the CNAs are detected in 2 ng or less of genomic DNA.

In some embodiments, the biological sample is a formalin-fixed paraffin-embedded tissue.

In some embodiments, the genes are two or more of CHD1, PTEN, CDKN1B, BRCA2, RB1, USP10, SERPINF1, TP53, SERPINB5 and MYC.

In some embodiments, the genes are three or more of CHD1, PTEN, CDKN1B, BRCA2, RB1, USP10, SERPINF1, TP53, SERPINB5 and MYC.

In some embodiments, the genes are four or more of CHD1, PTEN, CDKN1B, BRCA2, RB1, USP10, SERPINF1, TP53, SERPINB5 and MYC.

In some embodiments, the genes consist of only PTEN, MYC, CDKN1B and BRAC2.

In some embodiments, the methods disclosed herein may further comprise detecting the expression of one or more of the genes selected from the group consisting of: ABCC4 (NM_001105515.2), ACACA (NM_198834.1), ACSM1 (NM_052956.2), AMACR (NM_014324.4), APOC1 (NM_001645.5), APOE (NM_000041.2), ARHGEF26 (NM_001251963.1), BGN (NM_001711.3), BIRC5 (NM_001168.2), CDC20 (NM_001255.2), CDK1 (NM_001786.4), CENPF (NM_016343.3), COL10A1 (NM_000493.3), COL1A1 (NM_000088.3), DAPK1 (NM_001288729.1), DBNDD1 (NM_001042610.1), DLGAP5 (NM_014750.4), EPCAM (NM_002354.1), EZH2 (NM_001203247.1), FOXM1 (NM_202002.1), GJB1 (NM_000166.5), GLYATL1 (NM_001220494.1), GOLM1 (NM_016548.3), HMMR (NM_012484.2), HOXC4 (NM_014620.4), HOXC6 (NM_153693.3), HPN (NM_182983.1), MTHFD2 (NM_006636.3), MYBPC1 (NM_002465.2), MYO6 (NM_004999.3), NUSAP1 (NM_016359.4), PCA3 (NR_015342.1), PCAT7 (NR_121566.2), PCCB (NM_000532.4), PCLAF (NM_014736.5), PLA2G7 (NM_001168357.1), PRC1 (NM_199413.1), PYCR1 (NM_006907.2), RAB17 (NM_022449.3), RAP1GAP (NM_002885.1), RRM2 (NM_001034.1), SAPCD2 (NM_178448.3), SMPDL3B (NM_001009568.1), THBS2 (NM_003247.3), TK1 (NM_003258.1), TMEM238 (NM_001190764.1), TOP2A (NM_001067.3), TPX2 (NM_012112.4), UBE2C (NM_181803.1), ZWILCH (NM_017975.4), ABCF1, (NM_001090.2), G6PD (NM_000402.4), and TBP (NM_001172085.1).

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 shows the design of a blinded retrospective study with the number and criteria for inclusion/exclusion of the patients. BCR, biochemical recurrence; CNA, copy number alteration; MLPA, multiplex ligation-dependent probe amplification; PCa, prostate cancer; RP, radical prostatectomy.

FIG. 2 shows detection rates of CNAs in tumor DNA samples isolated from biopsy and surgery tissues.

FIG. 3 shows identification of both hemizygous deletion (marked by a light blue oval) and complete loss (marked by a dark blue oval) of PTEN and MYC amplification (marked by a red oval) in tumor DNA sample with 100% of formalin-fixed PC3 cells (A) or in DNA samples containing 50% of PC3 and 50% of RWPE cells (B) at 1 ng/reaction.

FIG. 4 shows high expression of mRNAs that are associated with poor survival time (disease-free survival).

FIG. 5 shows high expression of mRNAs that are associated with poor survival time (disease-free survival).

### DETAILED DESCRIPTION

Advanced genomic technology and bioinformatics have facilitated the discoveries of most, if not all, of the genes underlying the development of more than 30 major cancers. Different from other cancers, prostate tumors have the lowest rate of somatic single-nucleotide mutations/variations. In contrast, CNAs are more common in prostate tumors and are the major somatically acquired genetic aberrations. Although a number of studies have demonstrated the potential prognostic value of CNAs, DNA-based biomarkers have not been translated into a test platform for identification of higher-risk patients at diagnosis for personalized cancer care in clinical settings due to technical obstacles such as limited tumor tissue and contamination of normal tissues.

Advantageously, the present disclosure provides a panel of probes that is able to detect CNAs in a limited amount of tumor DNA that is readily obtainable from clinical specimens of diagnostic biopsy and radical prostatectomy surgery. Such CNAs detected at early disease stage (diagnosis or prostatectomy) predict the development of metastatic disease and prostate cancer-specific mortality. Additionally, the performance of CNAs to predict disease progression is independent of known clinicopathological variables. Moreover, the predictive performance is stronger in patients with clinically defined low-risk disease. This property is particularly useful clinically for predicting risk for disease progression at an early stage. Altogether, the findings disclosed herein support that the probe panel is feasible for clinical testing and can supplement clinicopathological variables to better distinguish between aggressive and indolent prostate cancer at the time of diagnosis, one of the most important clinical challenges in prostate cancer care. The CNVs detected with the methods provided herein may be used as a basis to take preventative measures to reduce a patient's cancer risk and to perform early, routine cancer screening.

As used herein, the term “subject” refers to an animal, including a mammal, such as a human being.

As used herein, a “patient” refers to a human subject.

As used herein, the singular forms “a,” “an” and “the” include plural referents unless the context clearly dictates otherwise. Thus, for example, reference to a compound, comprising “an extracellular domain” includes compounds with one or a plurality of extracellular domains.

As used herein, ranges and amounts can be expressed as “about” a particular value or range. About also includes the exact amount. Hence “about 5 bases” means “about 5 bases” and also “5 bases.”

As used herein, “normal range of coverage index” refers to a database or collection of data comprising information reflecting normal copy numbers for one or more genes. The index is generated based on information obtained from next generation sequencing (NGS) of biological samples using a set of probes wherein each probe in the set hybridizes a different segment of one or more genes. The segments can cover specific regions of a gene, or exons and exon-intron boundaries of one or more genes. The biological samples are obtained from patients known to have normal copy numbers for the one or more genes. That is, the patients have no deletions or duplications in the copy number of the one or more genes. The index can be generated from 1, 5, 10, 15, 20, 25, 50, 75, 100, or more biological samples. In a preferred embodiment, the database is generated from at least 100 biological samples. NGS is performed on the biological samples using the set of probes to obtain a sequence read for each probe. A normalization baseline is calculated from this information by adding the sequence reads from each probe in the set to obtain a total number of sequence reads for the set of probes and dividing the total number of sequence reads by the number of probes in the set of probes. The normalization baseline is used to calculate the coverage index for each probe, which is calculated by dividing the total number of sequence reads obtained for the probe by the normalization baseline.

As used herein, “established mean” refers to a mean calculated by adding the coverage indices for each probe and dividing by the total number of probes in the set of probes.

As used herein, “established standard deviation” refers to a value calculated using the established mean.

As used herein, “confidence interval” refers to a range of values defined so that there is a specified probability, also referred to as the confidence level, that the value of a parameter lies within it. Here, the confidence interval is calculated for each probe using the established mean and established standard deviation calculated from the normal range of coverage index using the following formula: Confidence interval=(established mean−2.57*established standard deviation, established mean+2.57*established standard deviation). The confidence interval can be based on a confidence level of 55%, 60%, 65%, 70%, 75%, 80%, 85%, 90%, 95%, or 99%. In a preferred embodiment, the confidence level is 99%.

As used herein, “normalization baseline” refers to a baseline value that is calculated by adding the sequence reads from each probe to obtain a total number of sequence reads for the set of probes and dividing the total number of sequence reads by the number of probes in the set of probes.

As used herein, “coverage index” refers to a value for a probe that is calculated by dividing a number of sequence reads obtained for the probe by the normalization baseline.

As used herein, the “probability value” or “p-value” is a value that is calculated for each probe based on the coverage index and the established mean and established standard deviation from the normal range of coverage index using the following equation: p value=2*(1−NORMSDIST(ABS(coverage index−established mean)/established standard deviation)). If the probability value is equal to or less than 10⁴, the biological sample has a CNV for the gene covered by the probe.

### Detection of a Gene CNV in a Biological Sample

Numerous technologies are available for analyzing DNA copy numbers in research settings, including fluorescent in situ hybridization (FISH) with various colors, quantitative polymerase chain reaction (PCR) with target and reference probes, comparative genomic hybridization (CGH) with different resolutions, and NGS. It has previously been impossible, however, to detect CNAs in somatic DNA in the clinical setting, likely due to lack of a practical, robust, and low-cost testing method/platform. For FISH, it is not practically feasible to analyze CNAs for a large number of targeted genes and chromosomal regions. CGH and NGS are expensive, technically demanding, and require larger amounts of tumor DNA (>100 ng) than what can be obtained from clinical specimens, especially from biopsy cores.

While PCR-based methods, including MLPA, require relatively small amounts of DNA and are more cost-effective, they also require careful probe design to control for variations and artifacts derived from common aneuploidy and heterogeneity of the tumor DNA. The existing commercially available mixed probes for MLPA are not suitable for detecting CNAs of prostate tumors because they are not specifically designed for this purpose. The probes for genes that are critical for prostate tumors are either not available or scattered across several different panels of mixed probes. More importantly, some reference probes in the mixed probes can also hybridize to genomic regions with DNA copy number changes due to common aneuploidy in prostate tumors and considerably affect the quality and reproducibility of CNA detection. Furthermore, commercially available probes for MLPA methods still require at least 50 ng/reaction, which makes it difficult for clinical testing of CNAs in prostate tumors, especially for measuring multiple targeted genes.

The probe panel disclosed herein overcome these drawbacks and provide consistently accurate measurements of CNAs. Using the information from a genome-wide CAN analysis of primary tumors in 1013 patients from five independent cohorts, CNAs associated with aggressive features were identified as well as genomic regions where CNAs were absent or rarely observed. Consistently low level of CNAs in reference regions across various types of samples is critical for increasing the accuracy and reliability of PCR-based testing. Improved sensitivity of the probe panel also enabled us to reduce the amount of DNA from 50 ng per reaction in standard MLPA to 2 ng per reaction. This advantage is particularly relevant for using leftover biopsy samples at diagnosis for prediction of clinical outcome. Furthermore, the inclusion of five probes per targeted gene that are distributed in different reactions in our four-color MLPA method also improves the reliability of detecting CNAs.

Ten genes were identified with CNAs that were associated with aggressive cancer, and five reference genomic regions where CNAs were absent or rarely found. A probe panel was then designed to measure CNAs in these 10 genes in tumor DNA isolated from formalin-fixed paraffin-embedded (FFPE) tissues.

Provided herein are methods of detecting gene CNVs in biological samples having one or more genes that can be used, for example, to assess a patient's risk of developing prostate cancer. Such method may comprise obtaining from the subject a biological sample from a prostate cancer; isolating genomic DNA from the biological sample; determining copy number alterations (CNAs) in one or more genes selected from the group consisting of: CHD1, PTEN, CDKN1B, BRCA2, RB1, USP10, SERPINF1, TP53, SERPINB5 and MYC in the genomic DNA; and classifying the subject as a progressor or a non-progressor based on the aforementioned determination.

In some embodiments, the methods disclosed herein may further comprise detecting the expression of one or more of the genes selected from the group consisting of: ABCC4 (NM_001105515.2), ACACA (NM_198834.1), ACSM1 (NM_052956.2), AMACR (NM_014324.4), APOC1 (NM_001645.5), APOE (NM_000041.2), ARHGEF26 (NM_001251963.1), BGN (NM_001711.3), BIRC5 (NM_001168.2), CDC20 (NM_001255.2), CDK1 (NM_001786.4), CENPF (NM_016343.3), COL10A1 (NM_000493.3), COL1A1 (NM_000088.3), DAPK1 (NM_001288729.1), DBNDD1 (NM_001042610.1), DLGAP5 (NM_014750.4), EPCAM (NM_002354.1), EZH2 (NM_001203247.1), FOXM1 (NM_202002.1), GJB1 (NM_000166.5), GLYATL1 (NM_001220494.1), GOLM1 (NM_016548.3), HMMR (NM_012484.2), HOXC4 (NM_014620.4), HOXC6 (NM_153693.3), HPN (NM_182983.1), MTHFD2 (NM_006636.3), MYBPC1 (NM_002465.2), MYO6 (NM_004999.3), NUSAP1 (NM_016359.4), PCA3 (NR_015342.1), PCAT7 (NR_121566.2), PCCB (NM_000532.4), PCLAF (NM_014736.5), PLA2G7 (NM_001168357.1), PRC1 (NM_199413.1), PYCR1 (NM_006907.2), RAB17 (NM_022449.3), RAP1GAP (NM_002885.1), RRM2 (NM_001034.1), SAPCD2 (NM_178448.3), SMPDL3B (NM_001009568.1), THBS2 (NM_003247.3), TK1 (NM_003258.1), TMEM238 (NM_001190764.1), TOP2A (NM_001067.3), TPX2 (NM_012112.4), UBE2C (NM_181803.1), ZWILCH (NM_017975.4), ABCF1, (NM_001090.2), G6PD (NM_000402.4), and TBP (NM_001172085.1).

The biological sample can be any biological tissue or fluid obtained from a subject including a human subject. Preferably, the biological sample is a cancer. The biological sample can also be from a tumor biopsy. Additionally, the biological sample can be a freshly collected sample. The biological sample can also be a formalin fixed paraffin embedded tissue sample. The biological sample can include tissues, cells, biological fluids and isolates thereof, including, but not limited to, cells or tumor cells isolated from body samples, such as, but not limited to, smears, sputum, biopsies, secretions, saliva, cerebrospinal fluid, bile, blood, plasma serum, lymph fluid, urine and feces, or tissue which has been removed from organs, such as breast, lung, intestine, skin, cervix, prostate, and stomach.

The biological sample can comprise one or more areas with different pathologic morphologies. Biological samples can comprise different pathologic morphologies in different locations within the sample. Some areas may have pathologic morphologies indicative of an aggressive form of cancer or malignancy. Other areas may show less aggressive pathologic morphologies. The biological sample may be obtained from one or more areas with the most aggressive pathologic morphology. The biological sample may also be obtained from one or more areas with the least aggressive pathologic morphology. The selection of targeted pathologic morphologies allows for targeted analysis of a biological sample. Alternatively, the biological sample may comprise several samples obtained from one or more of the different pathologic morphologies. In fact, samples for each different pathologic morphology present in a sample can be included in the biological sample, which would ensure a complete analysis of all possible sources of CNVs.

The biological sample can be obtained using known methods, including, but not limited to, needle biopsy, fine need aspiration, core biopsy, image-guided biopsy, CT-guided biopsy, ultrasound-guided biopsy, MRI-guided biopsy, aspiration biopsy, surgical biopsy, excisional biopsy, incisional biopsy, punch biopsy, vacuum-assisted biopsy, bone biopsy, liver biopsy, kidney biopsy, prostate biopsy, skin biopsy, liquid biopsy, endoscopic biopsy, laparoscopic biopsy, thoracoscopic biopsy, mediastinoscopic biopsy, laparotomy, thoracotomy, or sentinel lymph node mapping and biopsy.

The liquid biopsy can be an aspirate, blood, plasma serum, sputum, urine, or saliva. Preferably, the liquid biopsy is a blood sample. The liquid biopsy can be obtained using known methods, including, but not limited to, using known phlebotomy techniques, procedures, and methods. In an embodiment, the liquid biopsy is 10 mL of a blood sample that is collected in EDTA tubes. Preferably, the liquid biopsy is a blood sample or saliva sample. The biological sample may also be a fresh tissue sample. The biological sample may also be a solid tumor.

Once a biological sample is obtained, it can be preserved for further analysis using known methods. For example, the biological sample can be embedded in formalin-fixed paraffin to create a tissue block. The biological sample can also be frozen or fixed on a slide.

Exemplary CNVs include two or more of CHD1, PTEN, CDKN1B, BRCA2, RB1, USP10, SERPINF1, TP53, SERPINB5 and MYC. In an embodiment, the CNVs are PTEN, MYC, CDKN1B and BRAC2.

A CNV may be selected for inclusion in the methods disclosed herein based on its frequency of occurrence within a biological sample, since not every cell in the biological sample may contain the CNV. Instead, a CNV may appear in only a portion of the biological sample (e.g., some cells in the biological sample). Thus, a CNV may be selected for inclusion in the disclosed methods where its frequency in the biological sample is above a threshold amount including, for example, a percentage based on those cells that contain the CNV as compared to the total (or an estimated total) number of cells in the biological sample. For example, a CNV may be selected for inclusion in the methods where the frequency of the CNV is at least 5%, 10%, 15%, 20%, 25%, 30%, 35%, 40%, 45%, 50%, 55%, 60%, 65%, 70%, 75%, 80%, 85%, 90%, 95%, or 100% of the total number cells (e.g., estimated total number of cells) in the biological sample.

Further, the one or more CNVs included in the genetic profile may be selected to represent a particular pathologic morphology. Biological samples can comprise different pathologic morphologies in different locations within the sample. Some areas may have pathologic morphologies indicative of an aggressive form of cancer or malignancy. Other areas may show less aggressive pathologic morphologies. In some embodiments, CNVs are selected to represent the most aggressive pathologic morphologies in the biological sample. In other embodiments, mutations are selected that represent different (e.g., including all) pathologic morphologies.

Any known methods may be used to set an appropriate threshold level above which a CNV is determined to be present and below which a CNV is determined to not be present in a biological sample. For example, the threshold may be set at a certain number of copies of a CNV. Alternatively, the threshold may be set as a certain number of reads that detect the CNV including from next-generation sequencing (e.g., 10 or more reads). The threshold may also be set at a level of expression of the CNV.

The threshold may be set above the copy number of a CNV present in a control sample (e.g., a sample that does not contain the CNV). The threshold may also be set at an expression level present in a control sample not containing the CNV. In some embodiments, the expression level of a CNV must be at least 10%, 20%, 30%, 40%, 50%, 60%, 70%, 80%, 90%, 100% or greater than a control sample to determine that the CNV exists in the biological sample or liquid biopsy.

A number of methods can be used to detect the presence of CNVs in the biological sample and/or to quantitate the expression of those CNVs. Detection of CNVs can be performed at the protein level and/or the nucleic acid level. Those skilled in the art will appreciate that the methods indicated below represent some of the preferred ways in which the presence of CNVs can be detected and/or quantitated and in no manner limit the scope of the methods that can be employed. Those skilled in the art will also be able to determine operative and optimal assay conditions for each determination by employing routine experimentation. Such methods can include, but are not limited to, in situ hybridization (ISH), Western blots, ELISA, immunoprecipitation, immunofluorescence, flow cytometry, northern blots, Polymerase Chain Reaction (PCR), quantitative real-time PCR (qPCR), droplet digital PCR, immunocytochemistry (IHC), and next generation sequencing.

The expression level of the CNVs can be determined at the protein level or the nucleic acid level. Well known nucleic acid-based techniques for assessing expression can be used, including, but not limited to, determining the level of the mRNA for a gene in the biological sample, which method can use isolated RNA. Any RNA isolation technique that does not select against the isolation of mRNA can be used. Well known techniques for isolating RNA can also be used. The isolated mRNA from a biological sample can be used in hybridization or amplification assays, including, but not limited to, Southern or Northern analyses, PCR analyses, and probe assays.

An alternative method for determining the level of a CNV's mRNA in a biological sample or liquid biopsy involves the process of nucleic acid amplification, e.g., by RT-PCR (the experimental embodiment set forth in Mullis, 1987, U.S. Pat. No. 4,683,202) or digital PCR, ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. The above-described detection methods are useful for the detection of nucleic acid molecules that are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

Additionally, microarrays can be used to detect the expression of a CNV. In particular, DNA microarrays allow for the simultaneous measurement of the expression levels of large numbers of genes. Each array consists of a reproducible pattern of capture probes attached to a solid support. Labeled RNA or DNA may be hybridized to complementary probes on the array and then detected by laser scanning. Hybridization intensities for each probe on the array are determined and converted to a quantitative value representing relative gene expression levels (see, e.g., U.S. Pat. Nos. 6,040,138, 5,800,992, 6,020, 135, 6,033,860, and 6,344,316). High-density oligonucleotide arrays are particularly useful for determining the gene expression profile for a large number of RNAs in a sample.

Amplification-based assays can be used to measure a CNV. In such amplification-based assays, the corresponding nucleic acid sequence of a gene of interest acts as a template in an amplification reaction (for example, PCR). In a quantitative amplification, the amount of amplification product will be proportional to the amount of template in the original sample. Comparison to appropriate controls provides a measure of the copy number of the gene of interest, corresponding to the specific probe used. The presence of a higher level of amplification product, as compared to a control, is indicative of an amplified gene of interest.

Methods of “quantitative” amplification are well known to those skilled in the art. For example, quantitative PCR involves simultaneously co-amplifying a known quantity of a control sequence using the same primers. This provides an internal standard that may be used to calibrate the PCR reaction. Detailed protocols for quantitative PCR are provided, for example, in Innis et al. (1990) PCR Protocols, A Guide to Methods and Applications, Academic Press, Inc. N.Y.

Real-time PCR (RT-PCR) is another amplification technique that can be used to determine a CNV or levels of mRNA expression of a gene of interest. (See, e.g., Gibson et al., Genome Research 6:995-1001, 1996; Heid et al., Genome Research 6:986-994, 1996). Real time PCR evaluates the level of PCR product accumulation during amplification. This technique permits quantitative evaluation of mRNA levels in multiple samples. For gene copy levels, total genomic DNA is isolated from a sample. For mRNA levels, mRNA is extracted from tumor and normal tissue and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-106 copies of the gene of interest are generally sufficient. In addition, a standard curve is generated for the control sequence. This permits standardization of initial content of the nucleic acid of interest in a tissue sample to the amount of control for comparison purposes.

Droplet digital PCR is another amplification technique that can be used to determine CNVs or levels of mRNA expression of a gene of interest. This technique is a digital PCR method that utilizes a water-oil emulsion droplet system. A water-oil emulsion is used to form thousands of nanoliter-sized droplets that separate the template DNA molecules. PCR amplification takes place within each individual droplet. This allows thousands of individual amplifications to be measured within a single sample and also reduces the required sample size. Droplet digital PCR uses reagents and workflows similar to those used for most TaqMan probe-based assays, which are discussed below. After PCR, each droplet is analyzed or read to determine the number of droplets containing a PCR product in the original sample. That information is analyzed using Poisson statistics to determine the target template concentration in the original sample.

Methods of real-time quantitative PCR and digital droplet PCR using TaqMan probes are well known in the art. Detailed protocols for real-time quantitative PCR are provided, for example, for RNA in Gibson et al., 1996, A novel method for real time quantitative RT-PCR. Genome Res., 10:995-1001; and for DNA in Heid et al., 1996, Real time quantitative PCR. Genome Res., 10:986-994.

A TaqMan-based assay also can be used to quantify a particular genomic region.

TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification.

Other suitable amplification methods include, but are not limited to, ligase chain reaction (LCR) (see Wu and Wallace (1989) Genomics 4:560, Landegren et al. (1988) Science 241:1077, and Barringer et al. (1990) Gene 89:117), transcription amplification (Kwoh et al. (1989) Proc. Natl. Acad. Sci. USA 86:1173), self-sustained sequence replication (Guatelli et al. (1990) Proc. Nat. Acad. Sci. USA 87:1874), dot PCR, and linker adapter PCR, etc.

Fluorescence in situ hybridization (FISH) can also be used to determine the presence of a CNV in a biological sample. FISH is known to those of skill in the art (see Angerer, 1987 Meth. Enzymol., 152:649). Generally, in situ hybridization comprises the following major steps: (1) fixation of tissue or biological structure to be analyzed; (2) pre-hybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization; and (5) detection of the hybridized nucleic acid fragments.

In a typical in situ hybridization assay, cells or tissue sections are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions.

In some applications it is necessary to block the hybridization capacity of repetitive sequences. Thus, in some embodiments, tRNA, human genomic DNA, or Cot-1 DNA is used to block non-specific hybridization. Thus, in one embodiment of the present invention, the presence or absence of an amplification is determined by FISH.

The present disclosure also provides methods and materials for detecting a gene CNV in a biological sample having one or more genes. The methods may comprise: obtaining a set of probes for NGS wherein each probe in the set hybridizes a different segment of the one or more genes (e.g., genes associated with prostate cancer); performing NGS with the set of probes on the biological sample (e.g., a tissue sample from a prostate cancer) comprising the one or more genes to obtain a sequence read for each probe; creating a normalization baseline for a probe; generating a coverage index for a probe in the set of probes (e.g., by dividing the number of sequence reads obtained for the probe by the normalization baseline); and determining a difference between the coverage index of the probe and a set confidence interval established from a normal range of coverage index, wherein a CNV is detected where a p-value for the difference is equal to or less than a set threshold.

The present disclosure further provides methods and materials for detecting a gene CNV in a biological sample having one or more genes may also comprise: obtaining a set of probes for next generation sequencing wherein each probe in the set hybridizes a different segment of the one or more genes in the biological sample (e.g., genes associated with prostate cancer); performing NGS with the set of probes on the biological sample to obtain a sequence read for each probe; adding the sequence reads from each probe to obtain a total number of sequence reads for the set of probes; dividing the total number of sequence reads by the number of probes in the set of probes to generate a normalization baseline for a probe; determining a coverage index for the probe in the set of probes by diving a number of sequence reads obtained for the probe by the normalization baseline; and generating a p-value for a difference between the coverage index of the probe and a set confidence interval established from a normal range of coverage index, wherein a CNV is detected where the p-value is equal to or less than 10², 10³, or 10⁴.

A set of probes for next generation sequencing may be obtained based on the one or more genes for which a CNV is desired to be detected. The set of probes may comprise 1, 2, 3, 4, 5, 6, 7, 8, 9, 10, 15, 20, 25, 30, 35, 40, 45, 50 or more individual probes. The probes may be created to hybridize different segments of one or more genes associated with a known risk of prostate cancer. The probes may hybridize different segments of those genes, such as overlapping regions of exons, or exon-intron boundaries. The different segments may be of 1, 2, 3, 4, 5, 6, 7, 8, 9, 10, 15, 20, 25, 30, 35, 40, 45, 50, or more genes. The set of probes may be created using known methodologies, such as IDT in-silico technology.

Genomic DNA may be extracted from the biological sample using well-known conventional methods. A threshold amount of genomic DNA may be required for the disclosed methods, such as NGS. The threshold amount of genomic DNA can be 1 ng, 2 ng, 3 ng, 4 ng, 5 ng, 6 ng, 7 ng, 8 ng, 9 ng, 10 ng, 15 ng, 20 ng, 25 ng, 30 ng, 35 ng, 40 ng, 45 ng, or 50 ng.

Next generation sequencing is performed on the biological sample comprising one or more genes using the set of probes using known methodologies. From the next generation sequencing, a sequence read is obtained for each probe. Thus, the next generation sequencing provides the number of sequence reads for each probe as well as the aggregate number of sequence reads for the set of probes.

A normalization baseline is created for a probe. Such a normalization baseline may be created for each probe. The normalization baseline may be calculated by adding the number of sequence reads from each probe to obtain the total number of sequence reads for the set of probes and dividing that total by the number of probes in the set of probes. The normalization baseline may be used to generate a coverage index for a probe in the set of probes. A coverage index may be created for each probe. The normalization baseline and coverage index may be used to normalize the sequence read data obtained from next generation sequencing.

The normal range of coverage index may be determined by obtaining one or more biological samples having a normal copy number for each of the one or more genes; performing NGS on the one or more biological samples with the set of probes; adding the sequence reads from each probe to obtain a total number of sequence reads for the set of probes for each of the biological samples; dividing the total number of sequence reads for each of the biological samples by the number of probes in the set of probes to generate a normalization baseline for a probe; calculating a coverage index for each probe in the set of probes for the biological sample by dividing the number of sequence reads obtained for the probe by the normalization baseline; calculating an established mean and an established standard deviation for each probe using the coverage indices for the probes; and establishing a confidence interval for each probe using the established mean and the established standard deviation.

The normal range of coverage index may be used to establish a set confidence interval. The normal range of coverage index may comprise information reflecting normal copy numbers for one or more genes (e.g., normal copy numbers for genes associated with prostate cancer). The normal range of coverage index may be generated based on information obtained from NGS of biological samples known to have normal copy numbers of the one or more genes. Normal copy numbers of the one or more genes are copy numbers where there are no deletions are duplications. A set of probes wherein each probe in the set hybridizes a different segment of the one or more genes may be used to perform NGS. The segments may cover specific regions of a gene, or exons and exon-intron boundaries of one or more genes. The normal range of coverage index may be generated from information obtained from 1, 5, 10, 15, 20, 25, 50, 75, 100, or more biological samples. In one embodiment, the database is generated from at least 100 biological samples. NGS is used to obtain the number of sequence reads for each probe. That information is used to calculate a normalization baseline, which is done by adding the sequence reads from each probe to obtain a total number of sequence reads for the set of probes and dividing the total number of sequence reads by the number of probes in the set of probes. The normalization baseline is used to calculate the coverage index for each probe by dividing the total number of sequence reads obtained for the probe by the normalization baseline. The coverage index is used to calculate an established mean by adding the coverage indices for each probe and dividing by the total number of probes in the set of probes. The established mean is used to calculate an established standard deviation. The established mean and established standard deviation are used to calculate a set confidence interval.

A difference between the coverage index of a probe and a set confidence interval may be determined by calculating a p-value for the difference. The p-value may be calculated based on the coverage index, the established mean, and the established standard deviation. A CNV is detected where the p-value is equal to or less than a set threshold. The set threshold may be 10⁴.

A detected CNV may be an exon, intron, duplication (amplification), or deletion. The deletion may be heterozygous or homozygous.

The present disclosure further provides an electronic computer system that comprises one or more processors; and a memory storing one or more programs for execution by the one or more processors, the one or more programs comprising instructions for: analyzing data obtained from next generation sequencing of a biological sample having one or more genes using a set of probes, wherein the data comprises sequence reads for each probe; creating a normalization baseline for a probe; generating a coverage index for a probe in the set of probes; determining a difference between the coverage index of the probe and a set confidence interval established from a normal range of coverage index, wherein a copy number variant is detected where a p-value for the difference is equal to or less than a set threshold.

### Methods of Treating Prostate Cancer

The present disclosure also provides methods and materials for treating a patient with prostate cancer. Such methods may comprise obtaining from the subject a biological sample from a prostate cancer; isolating genomic DNA from the biological sample; determining copy number alterations (CNAs) in one or more genes selected from the group consisting of: CHD1, PTEN, CDKN1B, BRCA2, RB1, USP10, SERPINF1, TP53, SERPINB5 and MYC in the genomic DNA; and treating the subject with an anticancer agent based on the CNAs of the one or more genes.

CNAs detected by the probe panel have additional potential for guiding targeted therapy. Alterations of several genes on our DNA-based panel have been recently demonstrated to be associated with efficacy/sensitivity of drug treatment in prostate cancer. For example, prostate cancer with CHD1 deletion/SPOP-mutation was reported to be sensitive to abiraterone (Boysen G, Rodrigues D N, Rescigno P, et al. SPOP-mutated/CHD1-deleted lethal prostate cancer and abiraterone sensitivity. Clin Cancer Res. 2018; 24 (22): 5585-5593), while prostate cancer with PTEN loss responded better to treatment with ipatasertib and abiraterone than prostate cancer without PTEN loss (de Bono J S, De Giorgi U, Rodrigues D N, et al. Randomized Phase II study evaluating Akt blockade with ipatasertib, in combination with abiraterone, in patients with metastatic prostate cancer with and without PTEN loss. Clin Cancer Res. 2019; 25 (3): 928-936). Moreover, BRCA2 alterations in both germline and somatic DNA were found to be associated with improved response to treatment with PARP1/2 inhibitors (Mateo J, Carreira S, Sandhu S, et al. DNA-repair defects and olaparib in metastatic prostate cancer. N Engl J Med. 2015; 373 (18): 1697-1708; Mateo J, Porta N, Bianchini D, et al. Olaparib in patients with metastatic castration-resistant prostate cancer with DNA repair gene aberrations (TOPARP-B): a multicentre, open-label, randomised, phase 2 trial. Lancet Oncol. 2020; 21 (1): 162-174). Therefore, the model with CNAs of four genes (i.e., PTEN, MYC, CDKN1B and BRAC2) should provide additional information for treatment in patients with BRCA2 deletions.

In some embodiments, “treating” or “treatment” of a disease, disorder, or condition includes, at least partially, (1) preventing the disease, disorder, or condition, i.e., causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms. The treating or treatment of a disease or disorder may include treating or the treatment of cancer.

The term “treating cancer” refers to administration to a mammal afflicted with a cancerous condition and refers to an effect that alleviates the cancerous condition by killing the cancerous cells, but also to an effect that results in the inhibition of growth and/or metastasis of the cancer.

The anti-cancer therapy can include any well known therapies to treat cancer, including, but not limited to, surgical removal of the cancer, administration of chemotherapy, administration of radiation, administration of antibody therapies, and administration of anti-cancer drugs.

The term “chemotherapy” refers to the treatment of cancer or a disease or disorder caused by a virus, bacterium, other microorganism, or an inappropriate immune response using specific chemical agents, drugs, or radioactive agents that are selectively toxic and destructive to malignant cells and tissues, viruses, bacteria, or other microorganisms. Chemotherapeutic agents or drugs such as an anti-folate (e.g., Methotrexate) or any other agent or drug useful in treating cancer, an inflammatory disease, or an autoimmune disease are preferred. Suitable chemotherapeutic agents and drugs include, but are not limited to, actinomycin D, adriamycin, altretamine, azathioprine, bleomycin, busulphan, capecitabine, carboplatin, carmustine, chlorambucil, cisplatin, cladribine, crisantaspase, cyclophosphamide, cytarabine, dacarbazine, daunorubicin, doxorubicin, epirubicin, etoposide, fludarabine, fluorouracil, gemcitabine, hydroxyurea, idarubicin, ifosfamide, irinotecan, liposomal doxorubicin, lomustine, melphalan, mercaptopurine, Methotrexate, mitomycin, mitozantrone, oxaliplatin, paclitaxel, pentostatin, procarbazine, raltitrexed, steroids, streptozocin, taxol, taxotere, temozolomide, thioguanine, thiotepa, tomudex, topotecan, treosulfan, uft (uracil-tegufur), vinblastine, vincristine, vindesine, and vinorelbine.

This disclosure is further illustrated by the following examples which are provided to facilitate the practice of the disclosed methods. These examples are not intended to limit the scope of the disclosure in any way.

### EXAMPLES

### Example 1: General Methods

### Assay Design

Briefly, target CNAs and locations of probe sequences were identified by analyzing the raw data of genome-wide CNAs in the primary tumors of 1013 patients from five independent cohorts in public databases (Taylor B S, Schultz N, Hieronymus H, et al. Integrative genomic pro-filing of human prostate cancer. Cancer Cell. 2010; 18 (1): 11-22; Barbieri C E, Baca S C, Lawrence M S, et al. Exome sequencing identifies recurrent SPOP, FOXA1 and MED12 mutations in prostate cancer. Nature Genet. 2012; 44 (6): 685-689; Cancer Genome Atlas Research Network. The molecular taxonomy of primary prostate. Cancer Cell. 2015; 163 (4): 1011-1025; Liu W, Lindberg J, Sui G, et al. Identification of novel CHD1-associated collaborative alterations of genomic structure and functional assessment of CHD1 in prostate cancer. Oncogene. 2012; 31 (35): 3939-3948; Liu W, Xie C C, Thomas C Y, et al. Genetic markers associated with early cancer-specific mortality following prostatectomy. Cancer. 2013; 119:2405-2412). A total of 10 genes, including CHD1, PTEN, CDKN1B, BRCA2, RB1, USP10, SERPINF1, TP53, SERPINB5 and MYC, and five genomic regions where CNAs were absent or rarely observed were selected as targets of the probes.

Probe testing, validation, and development was carried out with a four-color multiplex ligation-dependent probe amplification (MLPA) in two separate phases. In the first phase, using formalin-fixed cell lines, PC3 (harboring PTEN deletion and MYC gain) and RWPE (no CNAs), the detection limits were defined for the amounts of input DNA and normal cell contamination to be 1 ng per reaction and 50%, respectively.

In the second phase, using DNA from fresh-frozen and FFPE tissues with known genome-wide CNAs identified by the Affymetrix SNP array 6.0 and the OncoScan CNV FFPE assay, a concordance of 98% and 96% was achieved, respectively, between the probe panel with four-color MLPA and the Affymetrix SNP 6.0 as well as the OncoScan.

### Study Subjects

A retrospective study was performed following Institution Review Board (IRB) approval. A total of 230 patients were selected with sufficient archival tissues from a large cohort of men undergoing surgery for treatment of clinically localized disease at Northshore University HealthSystem based on the following criteria: (a) men aged 18 and older; (b) diagnosed with PCa at the NorthShore University Health-System between 1 Jan. 2000 to 24 Aug. 2017; (c) tumor tissue greater than 2 mm in size available from prostate biopsy, prostatectomy, and/or transurethral resection of the prostate (TURP); (d) for living subjects: had a prostate biopsy, prostatectomy, and/or TURP at NorthShore between 1 Jan. 2000 to 1 Apr. 2011; (e) for deceased subjects: had a prostate biopsy, prostatectomy, and/or TURP at NorthShore between 1 Jan. 2000 to 24 August 2017. Out of the 230 patients, 121 developed metastatic disease, died from PCa, or had biochemical recurrence (BCR), and 109 had no evidence of disease at least 5 years after treatment.

### Blinded Study Design

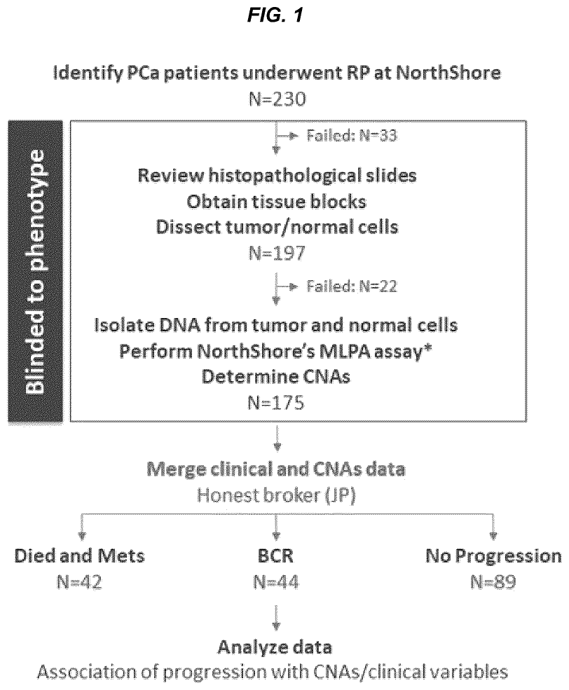

A blinded study design was employed to reduce potential observer bias in measuring CNAs (FIG. 1). First, eligibility of study subjects and disease outcomes were chart-reviewed by a designated study coordinator and honest broker (JP) via Epic electronic medical record software. Disease outcomes were kept confidential from all personnel involved in downstream sample processing and CNA detection. Second, histopathological slides were reviewed by two pathologists (JH and SC) to identify sufficient tumor (with ≥50% cancer cells) and matched normal tissues from the specimens. Histopathological features were blinded for the personnel involved in CNA data analyses. Among these 230 patients, 197 had sufficient tissues for macrodissection. Third, tissue specimens were then sent to the genomic laboratory for DNA isolation. GeneRead DNA FFPE Kit from Qiagen (Germantown, MD) was used for genomic DNA isolation following manufacturer's instructions with minor modifications. Sufficient DNA from 175 patients was available for the probe panel to detect CNAs. Fourth, the CNA calls were sent back to the study coordinator (JP) to merge with phenotypes. Finally, statistical analysis was performed.

Among the 175 patients, FFPE tissue specimens were obtained from diagnostic biopsy (N=97), radical prostatectomy (N=74), and TURP (N=7). Three patients had both biopsy and prostatectomy specimens. The clinical and demographic information of these patients are presented in Table 1.

TABLE 1
Clinical and demographic characteristics of prostate cancer patients

| | No progression | | Biochemical recurrence | | Lethal/metastatic | | |
|---|---|---|---|---|---|---|---|
| Variable | (n = 89) | | (n = 44) | | (n = 42) | | P value |
| Race, no. (%) of | 68 | (76.4) | 38 | (86.4) | 36 | (85.7) | .15 |
| Caucasians | | | | | | | |
| Age at diagnosis, mean | 61.0 | (56.0-65.0) | 62.0 | (57.8-68.3) | 67.0 | (61.0-73.8) | <2.2E−16 |
| (IQR), y | | | | | | | |
| PSA at diagnosis, mean | 4.72 | (4.05-6.50) | 6.22 | (4.90-7.91) | 5.38 | (4.15-9.78) | <2.2E−16 |
| (IQR), ng/mL | | | | | | | |
| Stage at biopsy/RP | | | | | | | |
| T1 | 1 | (1.12%) | 3 | (6.8%) | 4 | (9.5%) | .023 |
| T2 | 72 | (80.9%) | 25 | (56.8%) | 20 | (47.6%) | 5.88E−05 |
| T3 | 16 | (18.0%) | 15 | (34.1%) | 15 | (35.7%) | .018 |
| Missing | 0 | (0%) | 1 | (2.3%) | 3 | (7.1%) | . . . |
| Preoperative GG | | | | | | | |
| GG1 | 39 | (43.8%) | 14 | (31.8%) | 6 | (14.3%) | 8.52E−04 |
| GG2 | 23 | (25.8%) | 11 | (25.0%) | 2 | (4.8%) | .010 |
| GG3 | 19 | (21.3%) | 12 | (27.3%) | 18 | (42.9%) | .013 |
| GG4 | 3 | (3.4%) | 4 | (9.1%) | 8 | (19.0%) | .003 |
| GG5 | 5 | (5.6%) | 2 | (4.6%) | 8 | (19.0%) | .021 |
| Missing | 0 | (0%) | 1 | (2.3%) | 0 | (0%) | . . . |
| Postoperative GG | | | | | | | |
| GG1 | 17 | (19.1%) | 2 | (4.6%) | 0 | (0%) | 4.40E−04 |
| GG2 | 50 | (56.2%) | 22 | (50.0%) | 5 | (11.9%) | 6.70E−06 |
| GG3 | 14 | (15.7%) | 12 | (27.3%) | 10 | (23.8%) | .197 |
| GG4 | 1 | (1.1%) | 0 | (0%) | 1 | (2.4%) | .642 |
| GG5 | 2 | (2.3%) | 1 | (2.3%) | 8 | (19.0%) | 6.97E−04 |
| Missing | 5 | (5.6%) | 7 | (15.9%) | 18 | (42.9%) | . . . |
| Margin positive, no. (%) | 25 | (28.1%) | 19 | (43.2%) | 6 | (14.3%) | 0.26 |

Abbreviations: IQR, interquartile range; GG, grade group; PSA, prostate-specific antigen; RP, radical prostatectomy.

### Statistical Methods

I Baseline clinicopathological variables were compared among groups using Wilcoxon rank sum test for continuous variables or proportion trend test for categorical variables. Different rates of CNAs among groups were tested by the proportion trend test. Association between disease progression with CNAs and other clinicopathological variables was tested using univariable and multivariable logistic regression models. C-statistic, or area under the receiver operating curve (AUC), was used to estimate predictive performance for lethal/metastatic outcome. The difference between two C-statistics was compared using the DeLong test. All the statistical analyses were performed using R software (version 3.5.3). A two-tailed P<05 was considered statistically significant.

### Example 2: Detection of CNAs in Biopsy and Surgery Specimens

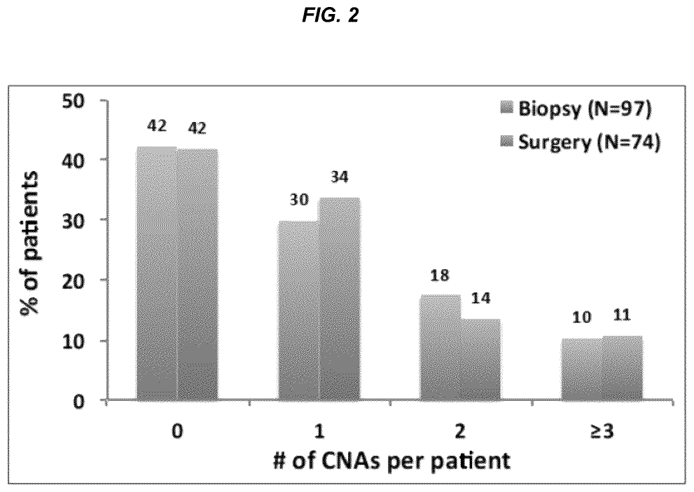

The probe panel detected CNAs in all 10 genes in somatic tumor DNA isolated from either biopsy or surgery tissues of the 175 PCa patients. Except for MYC where CNAs were in the form of copy number gains, CNAs in the other nine genes were in the form of copy number losses. The genes with greater than 10% CNAs among all patients were PTEN (16.6%), CHD1 (14.9%), RB1 (13.1%), USP10 (12.2%), and MYC (11.4%) (Table 2). Except for a weak co-occurrence of CNAs in MYC and RB1 (r²=17; P=1.54E-08), no substantial correlation was found for pairs of genes with CNAs (Table 6). When the 10 genes were considered together, 100 (57.1%), 45 (25.7%), and 14 (8.0%) patients had at least one, two, or three CNAs, respectively. The detection rate of CNAs was similar in DNA samples isolated from biopsy and surgery tissues, P>0.05 (FIG. 2).

TABLE 2
DNA copy number alterations (CNAs) by gene

| | | | | No. (%) CNAs | | | | | | |
|---|---|---|---|---|---|---|---|---|---|---|
| | | | | Non- | Biochemical | Lethal | | Univariable analysis | | |
| | | No. (%) | Type of | progression | recurrence | metastatic | | OR | | C-statistic |
| Variable | Chr | of CNAs | CNAs | (n = 89) | (n = 44) | (n = 42) | P trend | (95% CI) | P value | (95% CI) |
| PTEN | 10q23 | 29 | Deletion | 7 | 6 | 16 | 3.33E−05 | 7.21 | 9.46E−05 | 0.65 |
| | | (16.6) | | (7.9) | (13.6) | (38.1) | | (2.67-19.43) | | (0.57-0.73) |
| CHD 1 | 5q15 | 26 | Deletion | 12 | 5 | 9 | .303 | 1.75 | .25 | 0.54 |
| | | (14.9) | | (13.5) | (11.4) | (21.4) | | (0.67-4.55) | | (0.47-0.61) |
| RB1 | 13q14 | 23 | Deletion | 8 | 8 | 7 | .159 | 2.03 | .20 | 0.54 |
| | | (13.1) | | (9.0) | (18.2) | (16.7) | | (0.68-6.02) | | (0.47-0.60) |
| USP10 | 16q24 | 21 | Deletion | 11 | 5 | 5 | .919 | 0.96 | .50 | 0.50 |
| | | (12.0) | | (12.4) | (11.4) | (11.9) | | (0.31-2.96) | | (0.44-0.56) |
| MYC | 8q24 | 20 | Gain | 6 | 7 | 7 | .066 | 2.77 | .09 | 0.55 |
| | | (11.4) | | (6.7) | (15.9) | (16.7) | | (0.87-8.82) | | (0.49-0.61) |
| CDKN1B | 12p13 | 14 | Deletion | 5 | 4 | 5 | .203 | 2.27 | .22 | 0.53 |
| | | (8.0) | | (5.6) | (9.1) | (11.9) | | (0.62-8.32) | | (0.47-0.59) |
| BRCA2 | 13q12 | 13 | Deletion | 5 | 1 | 7 | .054 | 3.36 | .05 | 0.56 |
| | | (7.4) | | (5.6) | (2.3) | (16.7) | | (1.00-11.31) | | (0.49-0.62) |
| SERPINB5 | 18q21 | 10 | Deletion | 7 | 0 | 3 | .603 | 0.90 | .88 | 0.50 |
| | | (5.7) | | (7.9) | | (7.1) | | (0.22-3.67) | | (0.45-0.54) |
| TP53 | 17p13 | 9 | Deletion | 5 | 1 | 3 | .862 | 1.29 | .73 | 0.51 |
| | | (5.1) | | (5.6) | (2.3) | (7.1) | | (0.29-5.68) | | (0.46-0.55) |
| SERPINF1 | 17p13 | 1 | Deletion | 0 | 0 | 1 | .122 | | .99 | 0.51 |
| | | (0.6) | | | | (2.4) | | | | (0.49-0.54) |
| At least | | 100 | | 46 | 23 | 31 | .028 | 2.63 | .02 | 0.61 |
| one gene | | | | (51.7) | (52.3) | (73.8) | | (1.18-5.88) | | (0.53-0.70) |
| At least | | 45 | | 16 | 10 | 19 | .002 | 3.77 | 1.40E−03 | 0.64 |
| two genes | | | | (18.0) | (22.7) | (45.2) | | (1.67-8.50) | | (0.55-0.72) |
| At least | | 14 | | 4 | 3 | 7 | .022 | 4.25 | .03 | 0.56 |
| three genes | | | | (4.5) | (6.8) | (16.7) | | (1.17-15.44) | | (0.50-0.62) |

Abbreviations: CI, confidence interval; Chr, chromosome; OR, odds ratio.
′Compared between lethal/metastatic and non-progression.

### Example 3: Frequency of CNAs Among Progressors and Non-Progressors

Among the 175 patients, 89 had no evidence of disease progression after a follow-up of at least 5 years, 44 experienced BCR, and 42 had metastatic/lethal disease. CNAs were more common in patients with BCR or metastatic/lethal tumors than those without progression in 8 of the 10 genes (Table 2). More specifically, the frequencies of CNAs in patients with BCR were generally present at the frequencies between those found in patients without evidence of progression and patients with lethal/metastatic disease. Based on this observation and considering the uncertain prognostic potential of BCR, the remaining statistical analyses were limited to patients with more definitive clinical phenotypes: 89 patients without progression (non-progressors) and 42 patients with metastatic/lethal disease (progressors).

Progressors had significantly higher frequencies of CNAs when all 10 genes were considered as a group (Table 2). For example, 73.8% of progressor and 51.7% of non-progressors had at least one CNA in the 10 genes, odds ratio (OR)=2.63 (1.18-5.88); P=. 02. Similarly, 45.2% of progressors and 18.0% of non-progressors at least two CNAs, OR=3.77 (1.67-8.50); P=1.40E-03, and 16.7% of progressors and 4.5% of non-progressors had at least three CNAs, OR=4.25 (1.17-15.44); P=03. The association was stronger when CNAs of individual genes were compared. The gene with the largest difference in frequency of CNAs between progressors and non-progressors was PTEN, at 38.1% and 7.9%, respectively. Having CNAs at PTEN was associated with an OR (95% CI) of 7.21 (2.7-19.4) for metastatic/lethal disease, P=9.46E-05. Higher frequencies of CNAs in progressors than non-progressors were also found for seven additional genes, although the differences were not statistically significant (P>0.05). When CNAs of multiple individual genes were analyzed using a stepwise logistic regression analysis, three models performed similarly based on the Akaike information criterion: 150.41, 150.97, and 151.45 for the three-gene, four-gene, and five-gene model, respectively (Table 7). For each of these models, the number of patients with at least one CNA in the genes was tabulated and compared progressors to non-progressors. Next, each model's association with progression was tested and found that the four-gene model (PTEN, BRCA2, MYC, and CDKN1B) had the strongest association. Under this four-gene model, 64.3% of progressors and 22.5% of non-progressors had at least one CNA in these four genes, OR=6.21 (2.77-13.87); P=8.48E-06.

### Example 4: Association of Disease Progression with CNAs and Clinicopathological Variables

In addition to CNAs, several clinicopathologic variables were also associated with disease progression in univariate analysis, including age at diagnosis [OR=1.13 (1.07-1.20); P=1.19E-05], prostate-specific antigen (PSA) value at diagnosis [OR=5.6 (1.77-17.71); P=3.37E-03], clinical grade group at biopsy [OR=9.47 (3.87-23.18); P=8.46E-07] and clinical stage at biopsy [OR=2.85 (1.23-6.62); P=. 01] (Table 3). A positive surgical margin was not associated with disease progression, OR=0.73 (0.26-2.04); P=. 55. In a multivariable logistic regression analysis adjusting for the four implicated clinicopathological variables, CNAs of these four genes remained significantly associated with disease progression; OR=3.04 (1.11-8.33), P=. 03, suggesting that the association of CNAs was independent of known clinicopathological variables.

TABLE 3
Association of disease progression with CNAs and clinicopathological variables

| | Lethal | Non- | Univariable analysis | | Multivariable analysis | |
|---|---|---|---|---|---|---|
| | metastatic | progression | OR | | OR | |
| | (n = 42) | (n = 89) | (95% CI) | P value | (95% CI) | P value |
| Age dx, median | 67.0 | 61.0 | 1.13 | 1.19E−05 | 1.11 | 2.25E−03 |
| (IQR), y | (61.0-73.8) | (56.0-65.0) | (1.07-1.20) | | (1.04-1.18) | |

PSA at dx, ng/mL

| | | | | | | |
|---|---|---|---|---|---|---|
| PSA < 10 | 30 | 84 | Ref | | Ref | |
| | (71.4%) | (94.4%) | | | | |
| PSA ≥ 10 | 10 | 5 | 5.6 | 3.37E−03 | 4.14 | 0.06 |
| | (23.8% ) | (5.6%) | (1.77-17.71) | | (0.95-18.08) | |

Clinical GG at biopsy, no. (%)

| | | | | | | |
|---|---|---|---|---|---|---|
| GG ≤ 2 | 8 | 62 | Ref | | Ref | |
| | (19.0%) | (69.7%) | | | | |
| GG ≥ 3 | 33 | 27 | 9.47 | 8.46E−07 | 4.21 | 6.19E−03 |
| | (78.6%) | (30.3%) | (3.87-23.18) | | (1.50-11.80) | |

Clinical stage at biopsy, no (%)

| | | | | | | |
|---|---|---|---|---|---|---|
| T1 and T2 | 24 | 73 | Ref | | Ref | |
| | (57.1%) | (82.0%) | | | | |
| T3 | 15 | 16 | 2.85 | 0.01 | 2.16 | 0.14 |
| | (35 7%) | (18.0%) | (1.23-6.62) | | (0.77-6.07) | |

Surgical margin at RP, no. (%)

| | | | | | | |
|---|---|---|---|---|---|---|
| Negative | 20 | 61 | Ref | | | |
| | (47.6%) | (68.5%) | | | | |
| Positive | 6 | 25 | 0.73 | 0.55 | . . . | . . . |
| | (14.3%) | (28.1%) | (0.26-2.04) | | | |

CNAs at four genes, no. (%)

| | | | | | | |
|---|---|---|---|---|---|---|
| Negative | 15 | 69 | Ref | | Ref | |
| | (35.7%) | (77.5%) | | | | |
| Positive | 27 | 20 | 6.21 | 8.48E−06 | 3.04 | 0.03 |
| | (64.3%) | (22.5%) | (2.77-13.87) | | (1.11-8.33) | |

Abbreviations: CI, confidence interval; CNA, copy number alteration; dx, diagnosis; GG, grade group; OR, odds ratio; PSA, prostate-specific antigen; RP, radical prostatectomy; Ref, reference.

### Example 5: Performance of CNAs and Clinicopathological Variables for Predicting Disease Progression

TABLE 4
Performance of CNAs and clinicopathological variables for predicting disease

| Variable | AUC (95% CI) | Sensitivity | Specificity | PPV | NPV |
|---|---|---|---|---|---|
| Age at diagnosis | 0.74 (0.65-0.84) | . . . | . . . | . . . | . . . |
| PSA at diagnosis (≥10 vs <10 ng/mL) | 0.60 (0.52-0.67) | 0.25 | 0.94 | 0.67 | 0.74 |
| GG at biopsy (≥3 vs ≤2) | 0.75 (0.67-0.83) | 0.80 | 0.70 | 0.55 | 0.89 |
| Stage at biopsy (T3 vs T1/T2) | 0.60 (0.52-0.69) | 0.38 | 0.82 | 0.48 | 0.75 |
| CNAs at biopsy or surgery (four genes) | 0.71 (0.62-0.81) | 0.64 | 0.78 | 0.57 | 0.82 |
| In low-risk patients (GG ≤ 2 and | 0.69 (0.47-0.92) | 0.43 | 0.88 | 0.30 | 0.93 |
| PSA < 10), N = 65 | | | | | |
| In high-risk patients (GG ≥ 3 or | 0.59 (0.48-0.71) | 0.68 | 0.58 | 0.64 | 0.62 |
| PSA ≥ 10), N = 65 | | | | | |

Abbreviations: AUC, area under curve; CI, confidence interval; CNA, copy number alteration; GG, grade group; NPV, negative prediction value; PPV, positive prediction value; PSA, prostate-specific antigen.

The performance of CNAs to discriminate disease progression was comparable to other known clinicopathological variables (Table 4). The C-statistic (95% CI) of the four genes for discriminating metastatic/lethal disease from non-progression was 0.71 (0.62-0.81). In comparison, the C-statistic was 0.60 (0.52-0.67) for PSA value at diagnosis, 0.60 (0.52-0.69) for clinical stage at biopsy, 0.74 (0.65-0.84) for age at diagnosis, and 0.75 (0.67-0.83) for grade group.

Notably, the discriminative performance of CNAs in these four genes was better in patients with clinically low-risk disease (PSA at diagnosis <10 ng/ml and grade group≤2) than patients with clinically high-risk disease (PSA at diagnosis ≥10 ng/ml or grade group ≥3); C-statistic was 0.69 (0.47-0.92) and 0.59 (0.48-0.71), respectively. Specifically, among the seven progressors in the 65 patients with clinically low-risk disease, three (42.9%) had at least one CNA in the four genes. Having CNAs in these four genes was associated with an OR of 10.13 for progression, P=0.02 (Table 5). For patients with clinically high-risk disease, in comparison, having CNAs in these four genes was associated with an OR of 2.90 for progression, P=. 07.

TABLE 5
Association of CNAs at four genes with disease progression

| | | No. (%) of patients | | | |
|---|---|---|---|---|---|
| Clinical risk | CNAS | Non-progression | Lethal/metastatic | OR (95% CI) | P value |
| High risk^{a} | No | 18 (58.1) | 11 (32.4) | Ref | |
| | Yes | 13 (41.9) | 23 (67.6) | 2.9 (0.94-9.02) | .07 |
| Low risk^{b} | No | 54 (93.1) | 4 (57.1) | Ref | |
| | Yes | 4 (6.9) | 3 (42.9) | 10.13 (1.04-83.94) | .02 |

Abbreviations: CI, confidence interval; CNA, copy number alteration; OR, odds ratio; PSA, prostate-specific antigen; Ref, reference.
^{a}High-risk: PSA at diagnosis ≤10 ng/ml or grade group ≥3.
^{b}Low-risk: PSA at diagnosis <10 ng/mL and grade group ≤2.

Other measurements of the predictive performance of clinicopathological variables and CNAs were also estimated (Table 4). The performance of CNAs in general is comparable to other clinicopathological variables but differs among particular measurements. For example, the sensitivity of CNAs was higher than PSA but lower than grade group, while, conversely, the specificity of CNAs was higher than the grade group but lower than PSA.

TABLE 6
Pairwise correlation of CNAs among 10 genes
Correlation coefficient (R-square)

| | PTEN | CHD1 | RB1 | USP10 | MYC | CDKN1B | BRCA2 | SERPINB5 | TP53 | SERPINF1 |
|---|---|---|---|---|---|---|---|---|---|---|
| PTEN | — | 0.00 | 0.01 | 0.01 | 0.00 | 0.00 | 0.01 | 0.01 | 0.00 | 0.00 |
| CHD1 | — | — | 0.01 | 0.00 | 0.00 | 0.01 | 0.00 | 0.01 | 0.00 | 0.00 |
| RB1 | — | — | — | 0.01 | 0.03 | 0.00 | 0.17 | 0.00 | 0.00 | 0.00 |
| USP10 | — | — | — | — | 0.00 | 0.00 | 0.00 | 0.02 | 0.01 | 0.00 |
| MYC | — | — | — | — | — | 0.00 | 0.00 | 0.01 | 0.00 | 0.04 |
| CDKN1B | — | — | — | — | — | — | 0.01 | 0.01 | 0.00 | 0.07 |
| BRCA2 | — | — | — | — | — | — | — | 0.00 | 0.00 | 0.00 |
| SERPINB5 | — | — | — | — | — | — | — | — | 0.08 | 0.00 |
| TP53 | — | — | — | — | — | — | — | — | — | 0.00 |
| SERPINF1 | — | — | — | — | — | — | — | — | — | — |

Method: spearman,
CNAs: copy number alterations

TABLE 7
Stepwise regression analysis of CNAs with disease progression

| | | No. (%) subjects with any CNAs | | | | |
|---|---|---|---|---|---|---|
| | | Lethal metastasis | Non progression | OR | | C-statistic |
| Model | AIC | (n = 42) | (n = 89) | (95% CI) | P-value | (95% C1) |
| PTEN | 151.485 | 16 | 7 | 7.21 | 9.46E−05 | 0.65 |
| | | (38.1%) | (7.9%) | (2.67-19.43) | | (0.57-0.73) |
| PTEN, BRCA2 | 151.056 | 19 | 12 | 5.22 | 1.63E−04 | 0.65 |
| | | (45.2%) | (13.5%) | (2.60-13.77) | | (0.55-0.74) |
| PTEN, BRCA2, MYC | 150.41 | 24 | 17 | 5.56 | 2.91E−05 | 0.68 |
| | | (57.1%) | (19.1%) | (2.33-13.71) | | (0.59-0.78) |
| PTEN, BRCA2, MYC, | 150.97 | 27 | 20 | 6.21 | 8.48E−06 | 0.71 |
| CDKN1B | | (64.3%) | (22.5%) | (2.77-13.87) | | (0.62-0.81) |
| PTEN, BRCA2, MYC, | 151.45 | 29 | 30 | 4.33 | 3.11E−04 | 0.73 |
| CDKN1B, CHD1 | | (69.0%) | (33.7%) | (1.87-10.52) | | (0.63-0.83) |
| PTEN, BRCA2, MYC, | 152.97 | 30 | 35 | 3.82 | 1.18E−03 | 0.73 |
| CDKN1B, CHD1, TP53 | | (71.4%) | (39.3%) | (1.64-9.35) | | (0.63-0.83) |
| PTEN, BRCA2, MYC, | 154.96 | 30 | 38 | 3.32 | 3.92E−03 | 0.73 |
| CDKN1B, CHD1, TP53, | | (71.4%) | (42.7%) | (1.43-8.11) | | (0.63-0.83) |
| RB1 | | | | | | |
| PTEN, BRCA2, MYC, | 155.70 | 30 | 38 | 3.32 | 3.92E−03 | 0.73 |
| CDKN1B, CHD1, TP53, | | (71.4%) | (42.7%) | (1.43-8.11) | | (0.63-0.83) |
| RB1, SERPINF1 | | | | | | |
| All 10 genes | 159.224 | 31 | 46 | 2.62 | 0.03 | 0.74 |
| | | (73.8%) | (51.7%) | (1.11-6.52) | | (0.64-0.84) |

Lethal/metastasis: Patients developed metastasis or died of prostate cancer
Non progression: Patients without evidence of disease progression after >5 years of follow-up
AIC: Akaike information criterio,
OR: odds ration,
CI: confidence interval,
CNAs: copy number alterations

### Example 6: Targets of Multiplex Ligation-Dependent Probe Amplification (MLPA) and Probe Design

To accurately measure CNAs among large numbers of extremely heterogeneous prostate tumors, target probes need to reside in regions within genes that have been consistently deleted or amplified in the tumors of interest. In contrast, reference probes need to be located within regions where CNAs are absent or rarely observed. To achieve these goals in probe design, the raw data of genome-wide CNAs in the primary tumors of 1,013 patients from five independent cohorts in public databases was analyzed (Barbieri C E, Baca S C, Lawrence M S, Demichelis F, Blattner M, Theurillat J P, et al. Exome sequencing identifies recurrent SPOP, FOXA1 and MED12 mutations in prostate cancer. Nature genetics 2012; 44 (6): 685-9 doi 10.1038/ng.2279; Cancer Genome Atlas Research N. The Molecular Taxonomy of Primary Prostate Cancer. Cell 2015; 163 (4): 1011-25 doi 10.1016/j.cell.2015.10.025; Liu W, Lindberg J, Sui G, Luo J, Egevad L, Li T, et al. Identification of novel CHD1-associated collaborative alterations of genomic structure and functional assessment of CHD1 in prostate cancer. Oncogene 2012; 31 (35): 3939-48 doi 10.1038/onc.2011.554; Liu W, Xie C C, Thomas C Y, Kim S T, Lindberg J, Egevad L, et al. Genetic markers associated with early cancer-specific mortality following prostatectomy. Cancer 2013 doi 10.1002/cncr.27954; Taylor B S, Schultz N, Hieronymus H, Gopalan A, Xiao Y, Carver B S, et al. Integrative genomic profiling of human prostate cancer. Cancer cell 2010; 18 (1): 11-22 doi 10.1016/j.ccr.2010.05.026). A total of 10 genes that have been consistently shown to be associated with aggressive PCa or poor prognosis were selected for the probe design. These include CHD1 at 5q15, MYC at 8p21.2, PTEN at 10q23.31, CDKN1B at 12p13.1, BRCA2 at 13q13.1, RB1 at 13q14.2, USP10 at 16q24.1, SERPINF1 at 17p13.3, TP53 at 17p13.1, and SERPINB5 at 18q21.33. For most of these genes, five pairs of specific probes for each gene-except PTEN and BRCA2 with six pairs each and SERPINF1 with four pairs-were designed according to the UCSC Genome Browser hg38 assembly and synthesized by Integrated DNA Technologies (Coralville, Iowa). In addition, six reference probes were designed from different genomic regions where CNAs were absent or rarely observed.

### Example 7: Probe Testing, Validation and 4-Color MLPA Assay

In the first phase of probe testing, a probe mix containing five pairs for PTEN, five pairs for MYC, and six pairs of reference probes was made. To test these probe mixes under various conditions with different amounts of DNA, 50 ng/reaction of DNA was used from cell lines of prostate tumor and normal tissues as recommended by MRC-Holland (Amsterdam, Netherlands). Next, two batches of FFPE-equivalent DNA from prostate cell lines RWPE (normal control) and PC3 (cancer cell with PTEN deletion and MYC amplification) was created by fixing these cells with formalin using a common protocol for FFPE tissues. Samples of DNA isolated from these formalin-fixed cells of 100% RWPE, 100% PC3, 25% RWPE plus 75% PC3, 50% RWPE plus 50% PC3, and 75% RWPE plus 25% PC3 were used to test the detection limits with different amounts of normal DNA “contamination”. The detection limits were then tested with reduced amounts of input DNA from 50 ng to 0.2 ng per reaction using these types of DNA.

In the second phase of probe testing four separate probe mixes were made. The probes for each of these genes, except for SERPINB5, were distributed in different probe mixes to minimize the effect of variations in the assay. Each of the four separate probe mixes also contained five pairs of reference probes. A pair of universal primers labeled with four different fluorescent dyes (FAM, NED, PET and VIC) was designed to reduce the effects of cross contaminations among the four different reactions. 30 pairs of tumor and matched normal DNA were used with known CNAs identified by the Affymetrix SNP array 6.0 in fresh frozen prostate tumors to evaluate the performance of the probes. Next, the OncoScan CNV FFPE assay was used to further assess the performance of the probe panel by validating CNAs identified by OncoScan in nine selected genes using 2 ng of FFPE DNA. A mean concordance of 96% was achieved between the probe panel with 4-color MLPA and OncoScan for CNAs of the nine genes using a total of 16 different FFPE tumor-normal pairs.

### Example 8: Analytical Validity and Performance of a DNA-Based Probe Panel

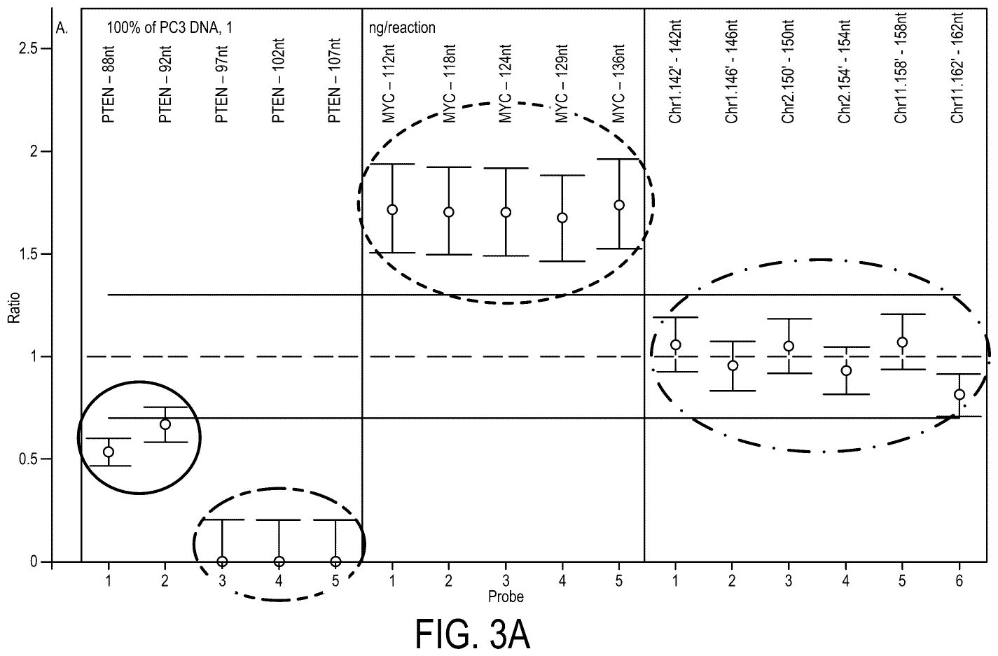

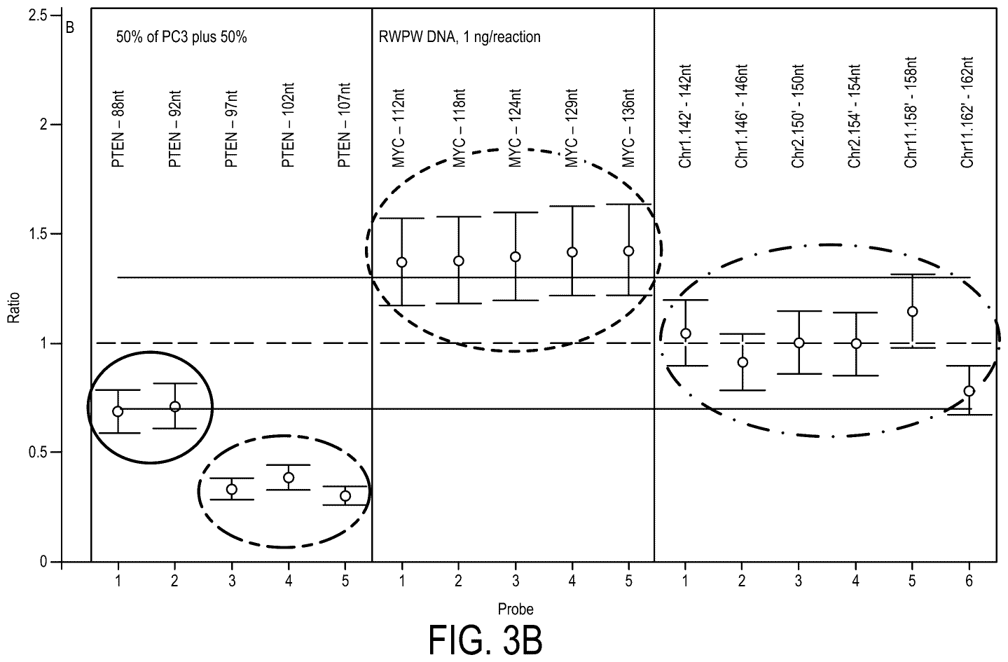

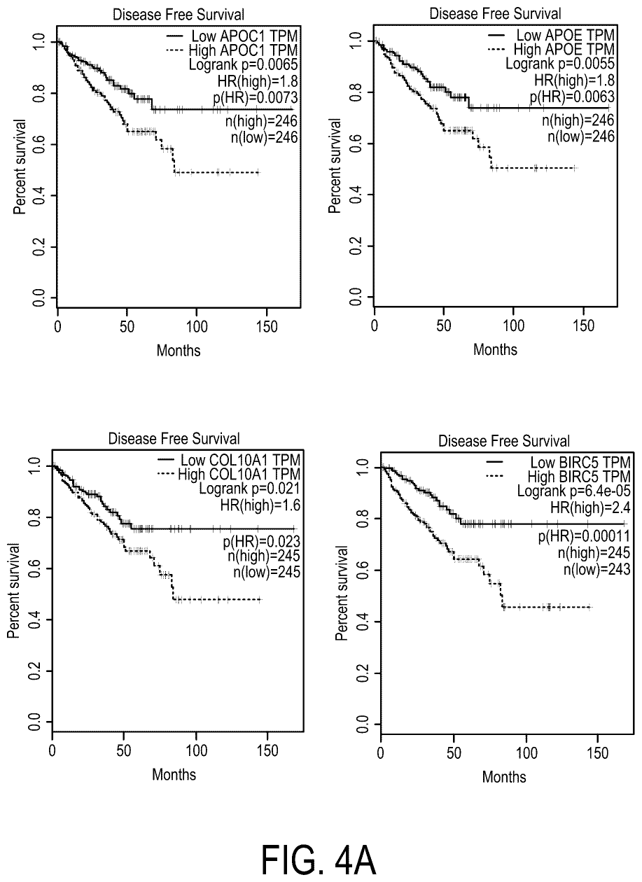

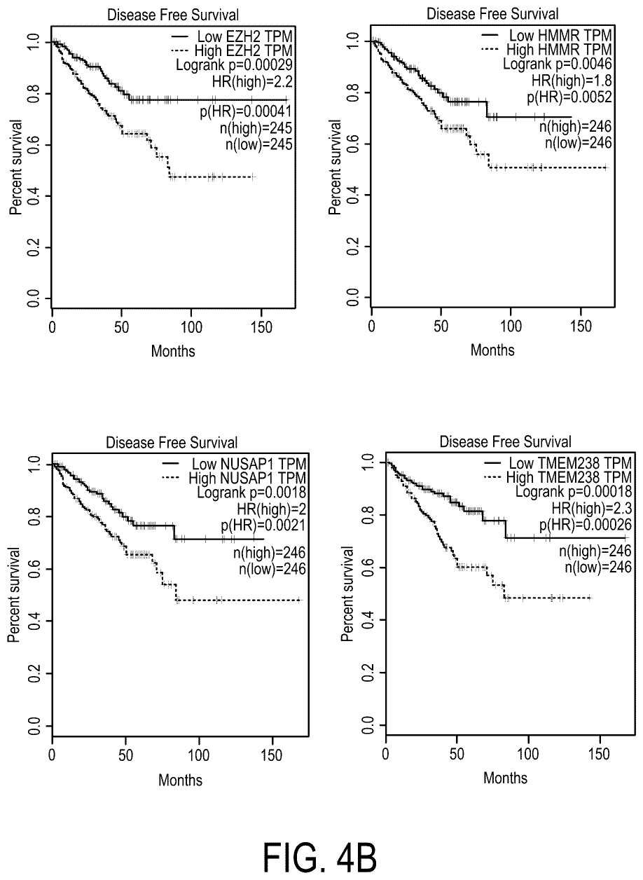

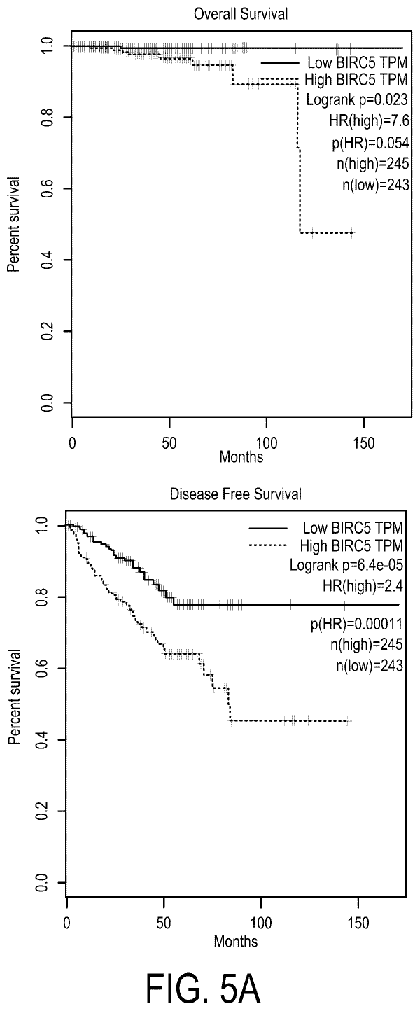

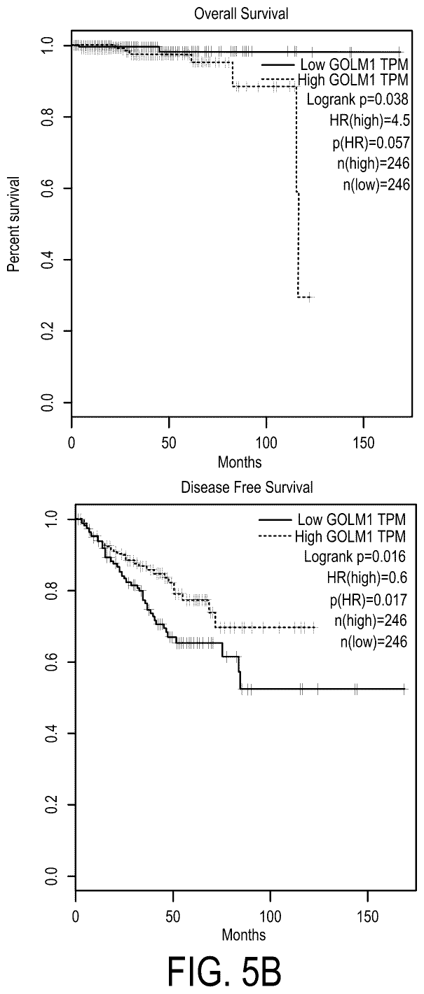

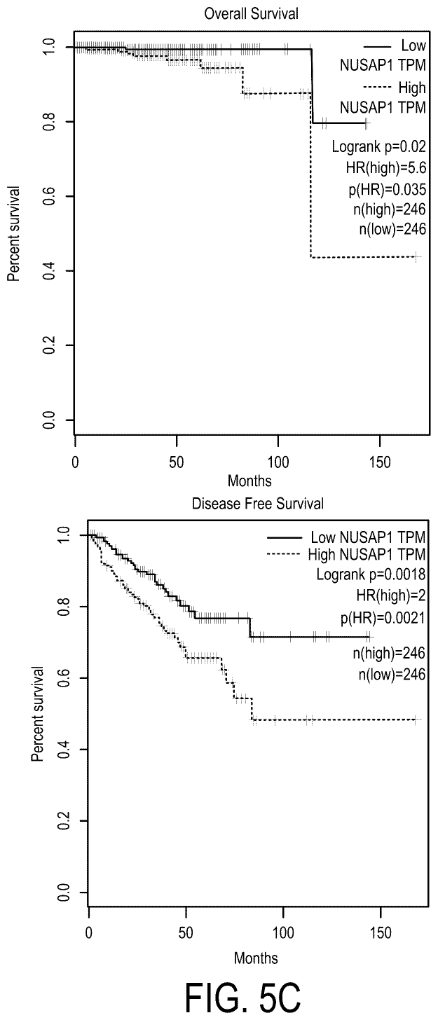

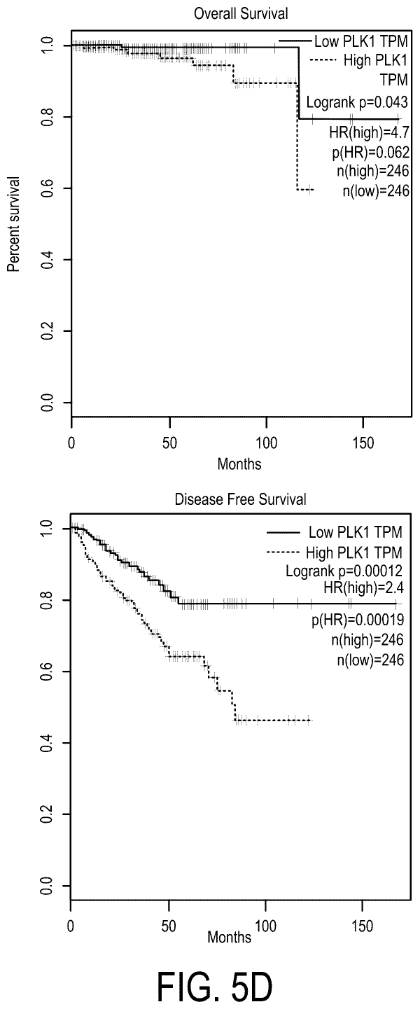

To evaluate the analytical performance of the DNA-based probe panel, genomic DNA from formalin-fixed PC3 cells was used with known hemizygous and homozygous deletions at PTEN and amplification of MYC. DNA from formalin-fixed RWPE cells without CNAs was used at these two genes as reference. The fragment sizes of the DNA isolated from formalin-fixed cell lines were found to be very similar to those isolated from FFPE tissues of the prostate. The detection limit for DNA amount was assessed by using amounts ranging from 50 ng/reaction (as recommended by MRC-Holland) to 0.2 ng/reaction (Table 8). As shown in FIG. 3A, the probe mix consistently identified both hemizygous deletion (marked by a light blue oval) and complete loss (marked by a dark blue oval) of PTEN and MYC amplification (marked by a red oval) in DNA amounts ≥1 ng/reaction with 100% DNA from formalin-fixed PC3 cells.

TABLE 8
Detection limit of DNA amount

| | | | F-PC3_1 | | F-PC3_2 | |
|---|---|---|---|---|---|---|
| Replication | Sample/reaction | | PTEN | MYC | PTEN | MYC |
| I | 0.2 | ng | d | g | d | g |
| | 0.4 | ng | d | g | d | g |
| | 0.8 | ng | d | g | failed | g |
| | 1 | ng | d | g | d | g |
| II | 0.2 | ng | d | g | d | g |
| | 0.4 | ng | d | g | d | g |
| | 0.8 | ng | d | g | d | g |
| | 1 | ng | d | g | d | g |
| I | 2 | ng | d | g | d | g |
| | 4 | ng | d | g | d | g |
| | 6 | ng | d | g | d | g |
| | 8 | ng | d | g | d | g |
| I | 10 | ng | d | g | d | g |
| | 20 | ng | d | g | d | g |
| | 30 | ng | d | g | d | g |
| | 40 | ng | d | g | d | g |
| | 50 | ng | d | g | d | g |

d = deletion,
n = no change,
g = gain

Next, three different concentrations of tumor/normal cell DNA were used at 0.4, 0.6 and 1 ng per reaction to determine the maximum amount of normal cell contamination tolerated in the identification of CNAs at PTEN and MYC (Table 9). As presented in FIG. 3B, both hemizygous deletion (marked by a light blue oval) and homozygous loss (marked by a dark blue oval) of PTEN and MYC amplification (marked by a red oval) were detected in DNA samples containing 50% PC3 and 50% RWPE cells at 1 ng/reaction. Under these conditions, the analytical specificity and reproducibility was 100% for heterozygous and/or homozygous deletions at PTEN and amplification of MYC using the DNA isolated from formalin-fixed cells. Therefore, it was determined to dissect tumor tissues containing ≥50% of cancer cells from biopsy and surgery specimens for downstream analysis.

TABLE 9
Detection limit of normal cell contamination

| | | F-PC3_1_0.4ng | | F-PC3_2_0.6ng | | F-PC3_3_1ng | |
|---|---|---|---|---|---|---|---|
| Replication | % tumor cell | PTEN | MYC | PTEN | MYC | PTEN | MYC |
| I | 25 | failed | g | failed | g | failed | g |
| | 50 | failed | g | d | g | d | g |
| | 75 | failed | g | d | g | d | g |
| | 100 | d | g | d | g | d | g |
| II | 25 | failed | g | failed | g | failed | g |
| | 50 | d | g | d | g | d | g |
| | 75 | d | g | d | g | d | g |
| | 100 | d | g | d | g | d | g |

d = deletion,
n = no change,
g = gain

The analytical validity of the probe panel and MLPA assay method using DNA isolated from both fresh-frozen and FFPE matched tumor/normal prostate tissues was assessed. As shown in Table 10, using genomic DNA from fresh-frozen tissues at 1 ng/reaction, a concordance of 98% between the probe panel and the Affymetrix SNP array 6.0 was achieved when detecting CNAs at PTEN and MYC. Considering the overall low quality of DNA from FFPE tissues, 2 ng/reaction was used in subsequent runs using the probe panel and assessed CNAs at nine genes in DNA from a total of 16 pairs of matched tumor-normal FFPE tissues. The DNA from these tissues was previously analyzed for genome-wide CNAs via the OncoScan CNV FFPE assay. Four separate experimental batches were performed and achieved a concordance of 94% to 100% with an average of 96% (Table 11). Finally, the probes were modified by eliminating the ones with inconsistent performance and added four probes of SERPINF1 to the final panel used in the downstream detection of CNAs in the tumors of 175 patients.

TABLE 10
Concordance calls of CNAs at PTEN and MYC between
the probe panel and the Affymetrix SNP array 6.0

| | PTEN | | MYC | |
|---|---|---|---|---|
| Tumor | | Array 6.0 | | Array 6.0 |
| ID | probe panel | CNAS | probe panel | CNAs |
| 172 | d | n | g | g |
| 196 | n | n | g | g |
| 241 | n | n | g | g |
| 259 | d | d | g | g |
| 372 | d | d | n | n |
| 406 | n | n | g | g |
| 522 | d | d | g | g |
| 535 | d | d | g | g |
| 541 | d | d | n | n |
| 667 | d | d | n | n |
| 682 | d | d | n | n |
| 733 | d | d | g | g |
| 759 | n | n | g | g |
| 780 | d | d | n | n |
| 795 | n | n | n | n |
| 810 | n | n | g | g |
| 823 | d | d | n | n |
| 848 | d | d | g | g |
| 913 | n | n | g | g |
| 926 | d | d | n | n |
| 981 | d | d | n | n |
| 982 | n | n | g | g |
| 1019 | d | d | g | g |
| 1038 | n | n | g | g |
| 1156 | d | d | n | n |
| 1422 | d | d | n | n |
| 1518 | d | d | n | n |
| 4441 | n | n | g | g |
| 4512 | d | d | n | n |
| 5384 | n | n | g | g |

CNAs: copy number alterations,
d = deletion;
n = no change;
g = gain

TABLE 11
Concordance calls of CNAs at 9 genes between the probe panel
with 4-color MLPA and the OncoScan FFPE CNV SNP array
Batch1

| 4C-MLPA/OncoScan | 75478 | 76122 | 76653 | PC-1T | Total |
|---|---|---|---|---|---|
| Deletion (#) | 3/3 | 4/4 | 3/3 | 6/4 | |
| Gain (#) | 2/2 | | 1/1 | 1/1 | |
| Normal (#) | 4/4 | 5/5 | 5/5 | 2/2 | |
| All (%) | 100 | 100 | 100 | 78 | 94 |

Batch2

| 4C-MLPA/OncoScan | 75483 | 79085 | 77263 | 77907 | Total |
|---|---|---|---|---|---|
| Deletion (#) | 4/4 | 5/5 | 2/2 | | |
| Gain (#) | | 2/2 | | | |
| Normal (#) | 5/5 | 2/2 | 7/7 | 9/9 | |
| All (%) | 100 | 100 | 100 | 100 | 100 |

Batch3

| 4C-MLPA/OncoScan | 75452 | 75547 | 79073 | 79076 | Total |
|---|---|---|---|---|---|
| Deletion (#) | 2/2 | 6/6 | 2/2 | 3/3 | |
| Gain (#) | 1/1 | 1/1 | | 1/1 | |
| Normal (#) | 5/6 | 3/2 | 7/7 | 5/5 | |
| All (%) | 89 | 89 | 100 | 100 | 94 |

Batch4

| 4C-MLPA/OncoScan | 76016** | 75564 | 77632 | 76659 | Total |
|---|---|---|---|---|---|
| Deletion (#) | 3/3 | 1/1 | 2/2 | 4/4 | |
| Gain (#) | | 1/1 | 1/1 | 1/1 | |
| Normal (#) | 5/6 | 7/7 | 6/6 | 4/4 | |
| All (%) | 89 | 100 | 100 | 100 | 97 |

CNAs: copy number alterations,
MLPA: multiplex ligation-dependent probe amplification,
CNV: copy number variation

### Example 9: Pilot Study to Evaluate the Performance of the DNA-Based Probe Panel

Before using a larger sample size, a blinded pilot study was performed with 21 progressor patients who died from PCa and 20 non-progressor patients. Genomic DNA was isolated from FFPE tissues from 33 biopsy, 5 transurethral resection of the prostate (TURP), and 3 surgery specimens. Among the 21 progressors, 6 (˜29%) had deletions at PTEN with another 3 (˜14%) harboring MYC gain (Table 12). Overall, ˜43% of the patients who died of PCa harbored CNAs of PTEN or MYC, while none of the non-progressors had CNAs in these two genes. The association between PCa mortality and CNAs of PTEN/MYC in this pilot study was statistically significant based on Fisher's exact test, with a positive prediction value of 100%.

TABLE 12
Pilot study with 21 progressors (lethal) and 20 non-progressors

| | All | Lethal | Non-lethal | PPV/NPV | P-value |
|---|---|---|---|---|---|
| PTEN_del | 6 | 6 | 0 | 100% | 0.021 |
| PTEN n | 35 | 15 | 20 | 57.14% | |
| % with del | 14.63% | 28.57% | 0 | | |
| | All | Lethal | Non-lethal | PPV/NPV | P-value |
| MYC_gain | 3 | 3 | 0 | 100% | 0.23 |
| MYC_n | 38 | 18 | 20 | 52.63% | |
| % with gain | 7.32% | 14.29% | 0 | | |
| | All | Lethal | Non-lethal | PPV/NPV | P-value |
| PTEN/ | 9 | 9 | 0 | 100% | 0.0013 |
| MYC_CNAS | | | | | |
| Without CNAs | 32 | 12 | 20 | 62.50% | |
| % with CNAs | 21.95% | 42.86% | 0 | | |

Fisher's exact test: The two-tailed P value,
PPV: positive prediction value,
NPV: negative prediction value,
del: deletion,
n: no change,
CNAs: copy number alterations

### Example 10: Cohort Description and DNA Isolation

This blinded retrospective study was performed following Institution Review Board (IRB) approval. A total of 230 patients were selected with enough archival tissues from a large cohort of men undergoing surgery for treatment of clinically-localized disease at NorthShore University HealthSystem based on the following criteria: 1) men aged 18 and older; 2) diagnosed with PCa at NorthShore University HealthSystem between Jan. 1, 2000 to Aug. 24, 2017; 3) tumor tissue ≥2 mm in size available from prostate biopsy, prostatectomy and/or TURP; 4) for living subjects: had a prostate biopsy, prostatectomy, and/or TURP at NorthShore between Jan. 1, 2000 to Apr. 1, 2011; 5) for deceased subjects: had a prostate biopsy, prostatectomy, and/or TURP at NorthShore between Jan. 1, 2000 to Aug. 24, 2017. Out of the 230 patients, 121 had biochemical recurrence (BCR), developed metastatic disease, and/or died from PCa, and 109 were non-progressors who have had no evidence of disease at least 5 years after treatment.

In this blinded study, patients were identified by a designated study coordinator and selected for clinical outcome data and availability of biopsy and prostatectomy specimens. The same coordinator served as the honest broker who de-identified patients' data and samples and kept disease outcomes confidential from all personnel involved in downstream sample processing and CNA data analyses. Histopathological slides were reviewed by two pathologists to identify enough tumorous (with ≥50% cancer cells) and matched normal tissues from the specimens. Among the 230 patients, 197 had enough tissues for macro-dissection (FIG. 1).

The GeneRead DNA FFPE kit from QIAGEN (Germantown, MD) was used for genomic DNA isolation following manufacturer's instructions with minor modifications. Briefly, the tissue specimens were centrifuged to the bottom of a 1.5 ml tube and added 160 μl of de-paraffinization solution to remove the wax. After 55 μl of nuclease-free water, 25 μl of Buffer FTB, and 20 μl of proteinase K were added, the samples were incubated at 56° C. for one hour or until the tissues were completely dissolved before incubating at 90° C. for another hour. Next, the lower clear phase was transferred into a new 1.5 ml tube, added 115 μl of nuclease-free water and 35 μl of Uracil-N-Glycosilase (UNG) to remove artificially-induced uracils by FFPE. After adding and mixing 2 μl of RNAs A, 250 μl of Buffer AL and 250 μl of ethanol, the lysate was transferred to the QIAamp MinElute column to bind the DNA and to remove residual contaminants using AW1, AW2 buffers and ethanol. Genomic DNA was eluted 2 times in ATE buffer with 20 μl each before the concentration was measured on a Qubit 3.0 Fluorometer. The quality was then assessed using gel electrophoresis.

Out of 197 patients, DNA from 22 patients did not pass quality control assessment. Samples from the remaining 175 patients were used for downstream CNAs analysis. These include 97 from biopsy, 7 from TURP and 74 from surgery specimens with 3 from both biopsy and surgery samples. Among these patients, 42 suffered from metastases or died from PCa, 44 had BCR, and 89 were non-progressors (FIG. 1). As shown in Table 1, the progressor patients were apparently older, had higher levels of pre-operational PSA, and higher Gleason grades in general than the non-progressors or the patients with BCR. The median follow-up after surgery among all patients was 10 years.

### Example 11: CNAs Assessment Using the Probe Panel with 4-Color MLPA

A basic MLPA method by MRC-Holland was used according to manufacturer's instructions using the own probe mixes and a 4-color MLPA reagent kit. Briefly, a unique set of all-synthetic probes was first hybridized to about 2 ng of FFPE DNA per reaction from each sample. Specific pairs of probes were then ligated to make a PCR template for high specificity. A set of fluorescent-labeled primers were next used for amplification of the templates. The PCR products were finally separated and quantified using the ABI-3500xl Genetic Analyzer. Quality control assessment was carried out through gel electrophoresis, the GeneMapper Software 5 and the Coffalyser software. CNAs in each samples was assessed using the Coffalyser software according to manufacturer's instructions.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A method for treating prostate cancer in a subject in need thereof comprising:
a.) obtaining from the subject a biological sample from a prostate cancer;
b.) isolating genomic DNA from the biological sample;
c.) determining copy number alterations (CNAs) in one or more genes selected from the group consisting of: CHD1, PTEN, CDKN1B, BRCA2, RB1, USP10, SERPINF1, TP53, SERPINB5 and MYC in the genomic DNA; and
d.) treating the subject with an anticancer agent based on the CNAs of the one or more genes in b).

2. The method of claim 1, wherein the CNAs are detected in less than 50 ng of genomic DNA.

3. The method of claim 1, wherein the CNAs are detected in less than 10 ng of genomic DNA.

4. The method of claim 1, wherein the CNAs are detected in 2 ng or less of genomic DNA.

5. The method of claim 1, wherein the biological sample is a formalin-fixed paraffin-embedded tissue.

6. The method of claim 1, wherein the genes are two or more of CHD1, PTEN, CDKN1B, BRCA2, RB1, USP10, SERPINF1, TP53, SERPINB5 and MYC.

7. The method of claim 1, wherein the genes are three or more of CHD1, PTEN, CDKN1B, BRCA2, RB1, USP10, SERPINF1, TP53, SERPINB5 and MYC.

8. The method of claim 1, wherein the genes are four or more of CHD1, PTEN, CDKN1B, BRCA2, RB1, USP10, SERPINF1, TP53, SERPINB5 and MYC.

9. The method of claim 8, wherein the genes consist of only PTEN, MYC, CDKN1B and BRAC2.

10. The method of claim 1 further comprising classifying the subject as a progressor or a non-progressor based on the determination in c).

11. A panel of probes for the prediction of aggressive prostate cancer, the panel comprising:
probes that specifically hybridize to copy number alterations (CNAs) in one or more genes selected from the group consisting of: CHD1, PTEN, CDKN1B, BRCA2, RB1, USP10, SERPINF1, TP53, SERPINB5 and MYC.

12. The panel of claim 11, wherein the genes are two or more of CHD1, PTEN, CDKN1B, BRCA2, RB1, USP10, SERPINF1, TP53, SERPINB5 and MYC.

13. The panel of claim 11, wherein the genes are three or more of CHD1, PTEN, CDKN1B, BRCA2, RB1, USP10, SERPINF1, TP53, SERPINB5 and MYC.

14. The panel of claim 11, wherein the genes are four or more of CHD1, PTEN, CDKN1B, BRCA2, RB1, USP10, SERPINF1, TP53, SERPINB5 and MYC.

15. The panel of claim 11, wherein the genes consist of only PTEN, MYC, CDKN1B and BRAC2.

16. A method for predicting prostate cancer progression in a subject in need thereof, the method comprising:
a.) obtaining from the subject a biological sample from a prostate cancer;
b.) isolating genomic DNA from the biological sample;
c.) determining copy number alterations (CNAs) in one or more genes selected from the group consisting of: CHD1, PTEN, CDKN1B, BRCA2, RB1, USP10, SERPINF1, TP53, SERPINB5 and MYC in the genomic DNA; and
d.) classifying the subject as a progressor or a non-progressor based on the determination in c).

17. The method of claim 16, wherein the prostate cancer is or will become metastatic and/or lethal if the subject is classified as a progressor.

18. The method of claim 16, wherein the CNAs are detected in 2 ng or less of genomic DNA.

19. The method of claim 16, wherein the biological sample is a formalin-fixed paraffin-embedded tissue.

20. The method of claim 16, wherein the genes are two or more of CHD1, PTEN, CDKN1B, BRCA2, RB1, USP10, SERPINF1, TP53, SERPINB5 and MYC.

21. The method of claim 16, wherein the genes are three or more of CHD1, PTEN, CDKN1B, BRCA2, RB1, USP10, SERPINF1, TP53, SERPINB5 and MYC.

22. The method of claim 16, wherein the genes are four or more of CHD1, PTEN, CDKN1B, BRCA2, RB1, USP10, SERPINF1, TP53, SERPINB5 and MYC.

23. The method of claim 22, wherein the genes consist of only PTEN, MYC, CDKN1B and BRAC2.
